(19) United States
(12) Patent Application Publication
(10) Pub. No.: US 2004/0087838 A1
(43) Pub. Date: May 6, 2004

(54) Meridian linking diagnostic and treatment system and method for treatment of manifested and latent maladies using the same

(76) Inventors: Mark Galloway, Riverton, UT (US); Joe Galloway, Saratoga Springs, UT (US)

Correspondence Address: Michael F. Krieger, KIRTON & McCONKIE, Suite 1800, 60 East South Temple, Salt Lake City, UT, 84111, US

(21) Appl. No.: 10/624,391

(22) Filed: Jul. 22, 2003

Related U.S. Application Data
(60) Provisional application No. 60/422,535, filed on Oct. 31, 2002.

(51) Int. Cl. G06F 19/00; G01N 33/48; G01N 33/50; A61B 5/00

(52) U.S. Cl. 600/300; 702/19

(57) ABSTRACT

The present invention features a computerized meridian linking diagnostic and treatment system that offers a new paradigm for practitioners in the EAV, GSR, EDS, and Meridian Stress Assessment fields. During the entire procedure, the present invention system outputs two permanent filters (frequencies) that link all of the body's meridians and stabilizes the data access points used for testing and carrying out the many functions of the present invention. The result is an interconnected meridian network linking the internal body systems to the data access points utilized by the system. The process begins by taking energetic readings at data access points. The computer stores the points that are the most stable. After the stable points are obtained, customized filters (frequencies) relating to specific issues or maladies (such as chemical toxins, allergies, digestion, etc.) are output or broadcast. Using only a single, but stable data access point as a reference point, if any of these filters creates a disturbance to any energetic component, cellular component, tissue, organ, or system of the body, each of which are linked by the interconnected meridian network, an imbalanced reading on the previously stable data access point will be created. The system will then automatically load products (remedies) that are useful for restoring homeostasis or balance. Each of the remedies are stored in the system database and can easily and quickly be scanned through until one or more products or remedies are discovered that will remove the underlying disturbance and allow the patient to obtain an improved level of health. The product/remedy is then placed in a holding tank that stores the results of each test. Specifically, the holding tank stores the filter(s) that created an imbalance/disturbance, the products (remedies) that allow the individual's body to restore homeostasis, balance, or improved health, and various prescription constraints that dictate administration of the products to the patient. The present invention also features several computer software functions, along with various methods of diagnosing maladies and treating a patient using an alternative medicine technique similar to a meridian stress assessment.

### RELATED APPLICATIONS

This application claims priority to U.S. Provisional Application Serial No. 60/422,535, filed Oct. 31, 2002, and entitled, “Diagnostic Meridian Linking System and Method.”

### BACKGROUND

1. Field of the Invention

The present invention relates to Galvanic Skin Response (GSR) or Electroacupuncture by Voll (EAV) devices and systems for diagnosing areas of imbalance as well as medical conditions existing within the body and for the homeopathic nutritional, or pharmaceutical treatment of these conditions, and particularly to a computerized electrodermal screening diagnostic and treatment system capable of linking the meridians of the body in a closed network accessible by a single data access or reference point, wherein the system allows for the diagnosing and homeopathic nutritional, or pharmaceutical treatment of both existing and latent maladies. The present invention further relates to computer software functions for enabling the same.

2. Background of the Invention and Related Art

Traditional medical science has long recognized certain electrical characteristics of human and other living organisms. For example, the traditional medical community has recognized the electrical potentials generated by the human body in such forms as brain waves as detected by electro-encephalographs (EEG), electrical impulses resulting from muscular heart activity as detected by electrocardiograms (EKG), and other electrical potentials measurable at other areas of the human body. While the relative levels of the electrical activity exhibit relatively small levels, such signals are nonetheless measurable and consistent.

In addition to measurable voltage levels, the human body as well as other mammalian organisms also comprise specific locations wherein the resistance value, and likewise the corresponding conductance value, are relatively predictable. These locations, or anatomical dermal conductance points, exhibit unique resistance values that vary greatly from all other locations on the human body. Interestingly, these locations on the body exhibit a resistive reading of approximately 100,000 ohms, for healthy tissue, and coincide with those locations on the body that correspond to the acupuncture points anciently defined by the Chinese. As is well known and documented, Chinese medical practitioners were aware of the art of treating unfavorable health conditions through the use of needles, which were used to pierce peripheral nerves to relieve pain. Although traditional acupuncture is still performed by some, today it is known that electrical stimulation of these points provides similar results.

The representative acupuncture points, and their relationship with organs and life systems of the human body have been characterized into more than 800 data access points. However, of these 800 points, 64 are recognized as particularly relevant to electroacupuncture. These 64 points are known as the Control Measurement Points. The data access points in the body are further organized into approximately 14 basic meridians. Such acupuncture points correspond to organs and systems such as the stomach, the small and large intestines, the bladder, the gall bladder, the heart, the lungs, the liver, the spleen/pancreas, the kidney, and the circulatory system. Studies indicate that many acupuncture points correspond to nerve innervations and trigger points. Most of the acupuncture points we use are generally located at the extremity region of the hands and are situated above major nerve trunks. Most have nerves within 0.5 centimeters of their location.

The measurable state of these acupuncture points reflects the condition of the related meridians, and therefore, the health of organs and other functions of the human body. As introduced above, the resistance value of healthy tissue at an acupuncture or conductance point generally is in the range of about 100,000 ohms. When such tissue is inflamed or infected, the conductivity is higher such that the measured resistance value appears lower than 100,000 ohms. Additionally, if the tissue is in a degenerative state, the conductivity is lower causing the resistance value to be higher. As such, this practice can be used to discover and diagnose various medical conditions existing within the body.

Several prior art systems have been developed to measure the resistance value of healthy or unhealthy tissue at the above described acupuncture points and return or indicate present resistive values to a clinician for use in diagnosing a present existing medical condition, as well as in prescribing a method and/or substance for treatment of the medical condition. Acupuncture points are located under the skin (epidermis) and are accessed electrically through the skin by using a probe or stylus having a tip pressed against the skin. The probe or stylus is attached to a diagnostic device capable of measuring the resistance value at any given location.

The practice described above utilizes electronic and electromechanical systems or devices commonly referred to as Electroacupuncture by Voll (EAV) devices, or Galvanic Skin Response (GSR) devices, wherein applied EAV or GSR is the practice of utilizing one of these systems to diagnose medical conditions in the body. Applied EAV or GSR provides not only an accurate holistic diagnosis very quickly, but also simultaneously the corresponding therapy to be used to treat the discovered medical condition. As the resistance value is discovered, various remedies can be tested to bring the resistance value back to normal, thus alleviating the medical condition.

Although present EAV/GSR devices or systems provide many advantages in the field of alternative medicine, several disadvantages or deficiencies are inherent therein. First, they require the testing of multiple data access points to test each meridian in the body, or each internal system or organ within the body. These systems look at each energetic meridian one at a time. In addition, they utilize 64 Control measurement points for testing. Second, they only look for and measure the meridians energetic balance, which is very time consuming. Third, they are only capable of treating existing or manifested maladies, or rather they are capable of only performing manifested malady treatments. Third, to find a remedy for a given malady, the user must perform a time consuming search through a list of manufactures and all of their product lists to find an appropriate and usable product. Other disadvantages and/or inherent deficiencies not specifically recited herein are known in the art.

### SUMMARY AND OBJECTS OF THE INVENTION

In light of the deficiencies in the prior art discussed above, the present invention seeks to advance alternative medicine treatment methods, and particularly treatment methods utilizing Galvanic Skin Response (GSR) and/or electro-acupuncture by Voll (EAV) devices.

In accordance with the invention as embodied and broadly described herein, the present invention features a method and system for linking the meridians of the body into an interconnected network that links all of the internal body systems and organs to the one or more data access points existing on an area of the body, namely the hands, wherein the selected data access points are only those of a “stable” nature, thus functioning as reference points, and for allowing the diagnosing and treatment of various manifested and dormant or latent maladies in a patient through a single reference point.

In one exemplary embodiment, the present invention features a computerized meridian linking EAV or GSR diagnostic and treatment system that offers a new paradigm for practitioners in the electroacupuncture and meridian stress testing fields, featuring ease of use, efficiency, speed, and practical clinical results. During the entire procedure, the present invention system outputs two permanent filters (frequencies) that link all of the body's meridians and stabilizes the data access points used for testing and taking measurements, particularly those located on the hands. The result is an interconnected network linking the internal body systems to the data access points utilized by the system. Once these points are established, the system broadcasts several temporary filters, such as a malady-specific customized filter, a products/remedies filter, a prescription constraint filter, a test plate filter, a homeopathic remedy filter, and an imprinting filter.

The process begins by taking energetic readings at data access points preferably located on the hands. The computer stores the points that are the most stable, which means those points whose capacitance readings are preferably between 45-55. After the meridians are linked and the stable points are obtained, one or more malady-specific customized filters (frequencies) relating to specific issues or maladies (e.g., chemical toxins, allergies, digestion problems, etc.) are output. If any of these filters creates a disturbance to any energetic component, cellular component, tissue, organ, or system of the body, an imbalanced reading on the previously stable data access point will be indicated. The system will then automatically load products (remedies) that are useful for restoring homeostasis or balance. Each of the remedies are stored in the system database within a products/remedies database and can easily and quickly be scanned through until one or more products or remedies are discovered that will remove the underlying disturbance and allow the patient to obtain an improved level of health. The product/remedy is then placed in a holding tank (also a database) that stores the results of each test. Specifically, the holding tank stores both the filter(s) that created an imbalance/disturbance and the products (remedies) that allow the individual's body to restore homeostasis, balance, or improved health. The holding tank also stores assigned prescription constraints and other associated rules as part of the product's electromagnetic signature.

If a filter (test) causes an imbalance the present invention system is capable of automatically loading those remedies that address or treat the issue, which remedies are made available for scanning. Automatic loading is unique to the present invention and provides significant advantages over prior art electrodermal screening (EDS), EAV or GSR devices.

Another unique feature of the present invention system is its ability to locate, define, and utilize a single stable data access point (defined as a reference point) for testing and treatment of any system or organ within the patient, or rather all of the bodies functions. Prior art EAV and GSR systems are incapable of this and require the testing of several data access points and numerous readings to test all of the bodies functions.

By linking all the energy meridians and associated organs and functions to a single data access point, or reference point, each component or function within the body (e.g., energetic, cellular, organ, system, or systemic interaction) can be tested to see if it responds adversely to the test. In other words, the present invention stresses the body and measures its response to that stress to both treat and diagnose any existing manifested or latent maladies. Prior art systems cannot test for a response to these five areas. Instead, prior art devices seek only to restore balance to the body by treating only manifested maladies.

The present invention induces a load or stress on these bodily components to see how they respond, to see their current function, and to see how they will respond in real life settings. As such, the present invention system is capable of detecting and treating both existing and dormant maladies, or rather the present invention is capable of performing both manifested illness treatments, as well as preventative treatments, while prior art devices are only capable of treating existing or manifested maladies or performing manifested illness treatments.

The present invention system comprises several computer-executed functions. One such function is the remedy scan function that employs a process of elimination to determine which of the products/remedies stored in a computerized database are the most appropriate for your client. The goal of the remedy scan function is to narrow a large list of potentials down to the single, most effective item for treatment.

Once a remedy/product is identified, further testing can be done through the stable reference point to determine the dilution and quantity/dosage of the remedy to be administered to the patient, as well as the duration of time the remedy is to be administered.

The present invention further features an imprinting function. Imprinting allows each electromagnetic signatures of items/remedies stored in the systems holding tank to be transferred to a liquid medium via the test plate. Electromagnetic signatures are stored in the database of the system as digital representations of the effect of an item on a biological entity. Imprinting stores the electromagnetic signatures between the carbon bonds of alcohol and water.

The present invention features various methods for controlling a computer to perform the functions and processes described herein, as well as several methods for operating the present invention system and for treating a patient using the present invention system.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates one exemplary embodiment of the computerized meridian linking electrodermal screening diagnostic system, wherein the components of the system are electrically coupled together;

FIG. 2 illustrates another exemplary embodiment of the computerized meridian linking electrodermal screening diagnostic system, wherein the components of the system are contained within a single device;

FIG. 3 illustrates, generally, the computerized meridian linking electrodermal screening diagnostic device and system according to one exemplary embodiment of the present invention;

FIG. 4 illustrates a block diagram of the hardware components of the meridian linking electrodermal screening diagnostic device according to one exemplary embodiment of the present invention;

FIG. 5 illustrates a user interface screen-shot of a client or patient information setup page according to one exemplary embodiment of the present invention;

FIG. 6-A illustrates a simple circuit diagram of the inner workings of the body's electronic function as proposed by the present invention;

FIG. 6-B illustrates an equivalent circuit diagram depicting the network of interconnected systems and organs of the body as proposed by the present invention;

FIG. 7 illustrates a flow chart of the meridian linking and data access point stabilizing computer software function according to one exemplary embodiment of the present invention;

FIG. 8 illustrates an exemplary user interface screen-shot of some of the features and functions of the computerized meridian linking electrodermal screening diagnostic system;

FIG. 9 illustrates a user interface screen-shot featuring activation of the stable point range modification function of the computerized meridian linking electrodermal screening diagnostic system;

FIG. 10 illustrates a flow chart of the computer software Filter Testing Function designed to output a stressing filter to the body of the patient;

FIG. 11 illustrates a user interface screen-shot featuring the list of available filters or frequencies stored in the filter database and accessible by the computerized meridian linking electrodermal screening diagnostic system, according to one exemplary embodiment of the present invention;

FIG. 12 illustrates a user interface screen-shot featuring activation of the manual testing function of the computerized meridian linking electrodermal screening diagnostic system, according to one exemplary embodiment of the present invention;

FIG. 13 illustrates a flow chart of the computer software Remedy Scan Function according to one exemplary embodiment of the present invention;

FIG. 14 illustrates a user interface screen-shot featuring activation of the automatic filter testing function of the computerized meridian linking electrodermal screening diagnostic system, according to one exemplary embodiment of the present invention;

FIG. 15 illustrates a user interface screen-shot featuring activation of the stable point maintenance function of the computerized meridian linking electrodermal screening diagnostic system, according to one exemplary embodiment of the present invention;

FIG. 16 illustrates of flow chart of the amplification computer software function according to one exemplary embodiment of the present invention;

FIG. 17 illustrates a user interface screen-shot featuring activation of the amplification function of the computerized meridian linking electrodermal screening diagnostic system, according to one exemplary embodiment of the present invention;

FIG. 18 illustrates a flow chart of the remedy dilution computer software function according to one exemplary embodiment of the present invention;

FIG. 19 illustrates a flow chart of the remedy type/dosage computer software function according to one exemplary embodiment of the present invention;

FIG. 20 illustrates a flow chart of the remedy duration computer software function according to one exemplary embodiment of the present invention;

FIG. 21 illustrates a user interface screen-shot featuring activation of the type/dosage function of the computerized meridian linking electrodermal screening diagnostic system, according to one exemplary embodiment of the present invention;

FIG. 22 illustrates a user interface screen-shot featuring activation of the manual remedy search function of the computerized meridian linking electrodermal screening diagnostic system, according to one exemplary embodiment of the present invention;

FIG. 23 illustrates a user interface screen-shot featuring activation of the holding tank of the computerized meridian linking electrodermal screening diagnostic system, according to one exemplary embodiment of the present invention, wherein the holding tank may contain detailed descriptions of the remedies contained therein;

FIG. 24 illustrates a flow chart of the test plate computer software function according to one exemplary embodiment of the present invention;

FIG. 25 illustrates a user interface screen-shot featuring activation of the test plate function of the computerized meridian linking electrodermal screening diagnostic system, according to one exemplary embodiment of the present invention;

FIG. 26 illustrates a flow chart of the imprinting computer software function according to one exemplary embodiment of the present invention;

FIG. 27 illustrates a flow chart of one exemplary method used by a clinician to link the meridians of the body and establish one or more stable reference points using the computerized meridian linking electrodermal screening diagnostic system of the present invention;,

FIG. 28 illustrates a flow chart of one exemplary method used by a clinician to diagnose one or more existing or latent medical conditions or maladies in the body using the computerized meridian linking electrodermal screening diagnostic system of the present invention;

FIG. 29 illustrates a flow chart of one exemplary method used by a clinician to stress the physiology of the patient to reveal latent or unmanifested maladies using the computerized meridian linking electrodermal screening diagnostic system of the present invention;

FIG. 30 illustrates a flow chart of one exemplary method used by a clinician to determine the dilution, type/dosage, and duration of one or more remedies to be administered to the patient using the computerized meridian linking electrodermal screening diagnostic system of the present invention; and

FIG. 31 illustrates a flow chart of one exemplary method used by a clinician to imprint the electromagnetic signatures of one or more remedies into a homeopathic solution for later administration to the patient using the computerized meridian linking electrodermal screening diagnostic system of the present invention.

### DETAILED DESCRIPTION OF THE PREFERRED EMBODIMENTS

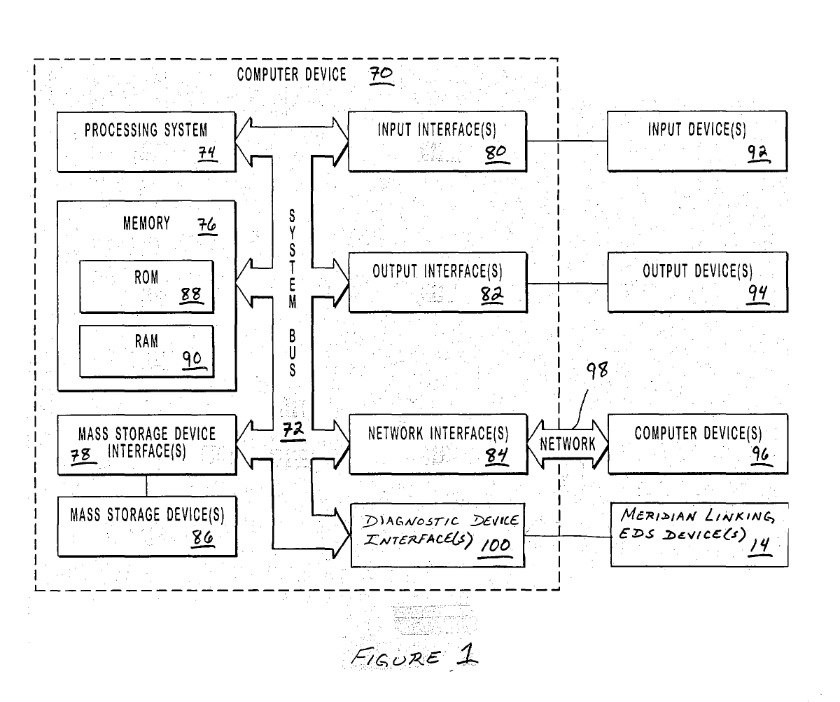

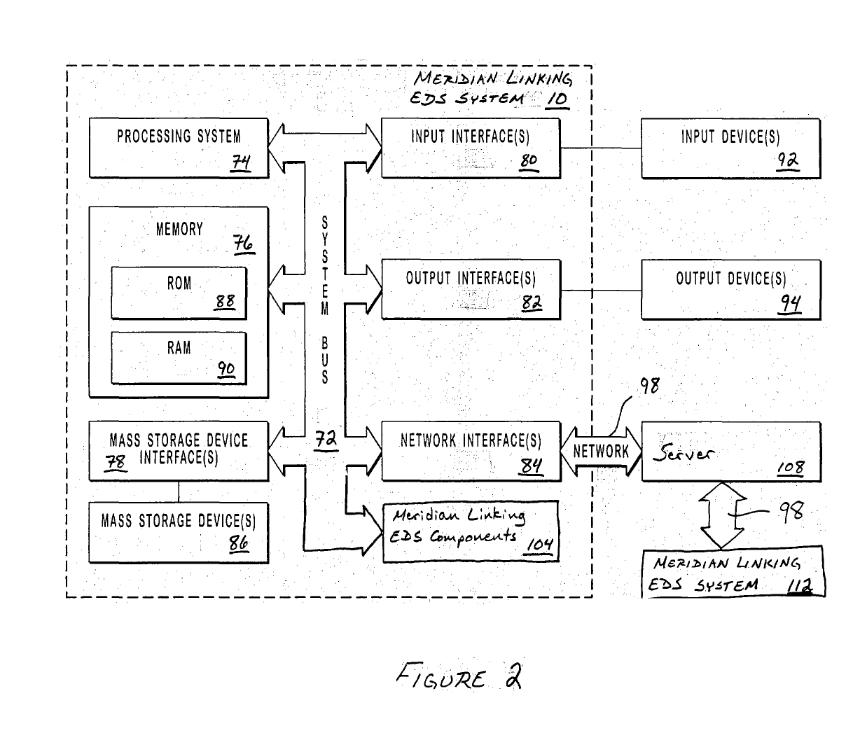

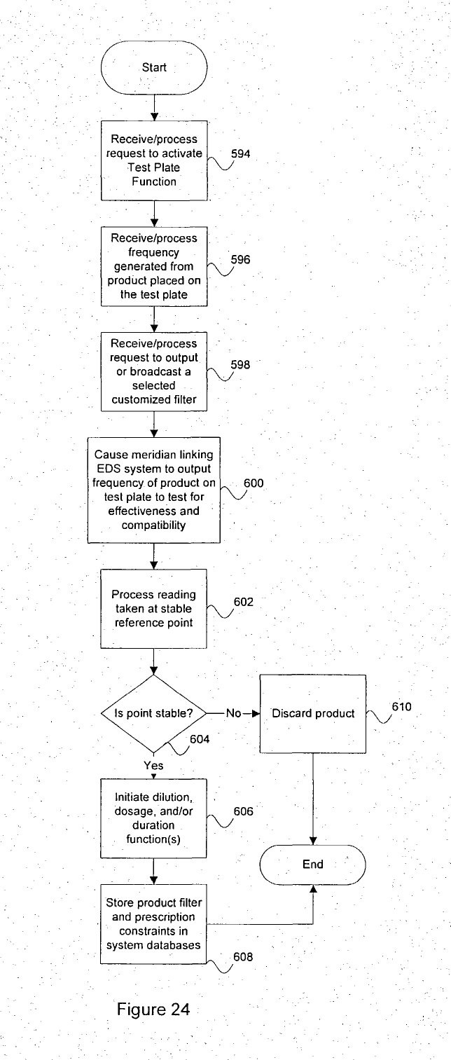

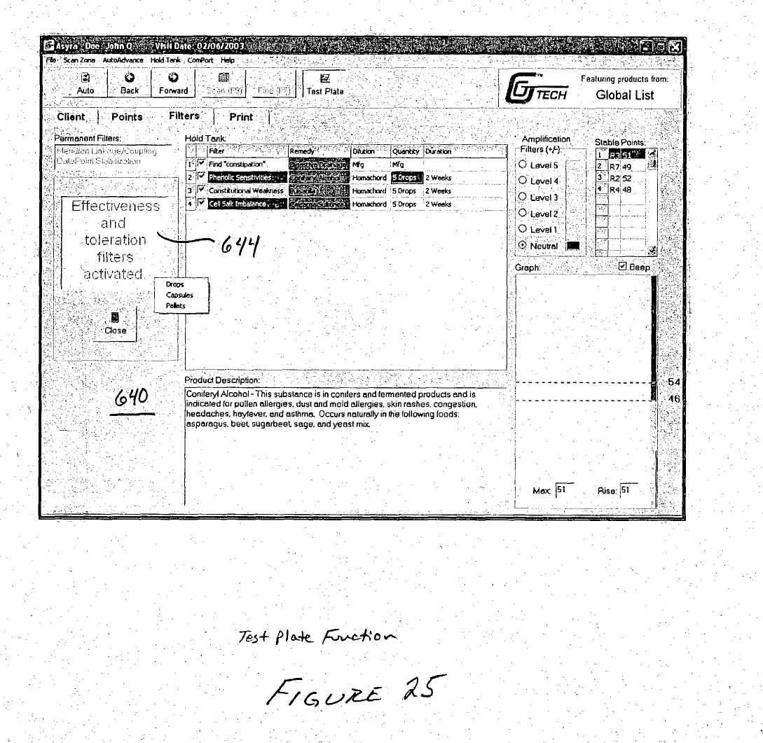

It will be readily understood that the components of the present invention, as generally described and illustrated in the figures herein, could be arranged and designed in a wide variety of different configurations. Thus, the following more detailed description of the embodiments of the system and method of the present invention, and represented in FIGS. 1 through 31, is not intended to limit the scope of the invention, as claimed, but is merely representative of the presently preferred embodiments of the invention.

The presently preferred embodiments of the invention will be best understood by reference to the drawings wherein like parts are designated by like numerals throughout.

The following disclosure of the present invention is grouped into several subheadings for convenience of the reader. First, a detailed description of an exemplary computerized operating environment will be set forth. Second, a general discussion of meridians, acupoints, and various Meridian Stress Assessment (MSA) testing systems is presented. Third, the present invention meridian linking diagnostic device and system is detailed and explained. Fourth, the individual software functions enabling the system to operate are detailed and set forth. Fifth, several methods of operating the diagnostic device and system are presented, along with methods of treating various existing and latent maladies or medical conditions within the body. The utilization of the above-mentioned subheadings are for discussion purposes only and should not be construed as limiting in any sense.

### Exemplary Computerized Operating Environment

FIG. 1 and the corresponding discussion is intended to provide a general description of a suitable operating environment in which the invention may be implemented. One skilled in the art will appreciate that the invention may be practiced by one or more computing devices and in a variety of system configurations, including and preferably within a networked configuration.

Embodiments of the present invention embrace one or more computer readable media, wherein each medium may be configured to include or includes thereon data or computer executable instructions for manipulating data. The computer executable instructions include data structures, objects, programs, routines, or other program modules that may be accessed by a processing system, such as one associated with a general-purpose computer capable of performing various different functions or one associated with a special-purpose computer capable of performing a limited number of functions. Computer executable instructions defined by and existing within programmable computer code cause the processing system to perform a particular function or group of functions and are examples of program code means for implementing steps for methods disclosed herein. Furthermore, a particular sequence of the executable instructions provides an example of corresponding acts that may be used to implement such steps.

Examples of computer readable media include random-access memory (“RAM”), read-only memory (“ROM”), programmable read-only memory (“PROM”), erasable programmable read-only memory (“EPROM”), electronically erasable programmable read-only memory (“EEPROM”), compact disk read-only memory (“CD-ROM”), or any other device or component that is capable of providing data or executable instructions that may be accessed by a processing system.

With reference to FIG. 1, a representative computer and computer system for implementing the invention shown in FIG. 1 includes computer device 70, which may be a general-purpose or special-purpose computer. For example, computer device 70 may be a personal computer, a notebook computer, a personal digital assistant (“PDA”) or other hand-held device, a workstation, a minicomputer, a mainframe, a supercomputer, a multi-processor system, a network computer, a processor-based consumer electronic device, or the like.

Computer device 70 includes system bus 72, which may be configured to connect various components thereof and enables data to be exchanged between two or more components. System bus 72 may include one of a variety of bus structures including a memory bus or memory controller, a peripheral bus, or a local bus that uses any of a variety of bus architectures. Typical components connected by system bus 72 include processing system 74 and memory 76. Other components may include one or more mass storage device interfaces 78, input interfaces 80, output interfaces 82, network interfaces 84, and/or EDS diagnostic device interfaces 100, each of which will be discussed below.

Processing system 74 includes one or more processors, such as a central processor and optionally one or more other processors designed to perform a particular function or task. It is typically processing system 74 that executes the instructions provided on computer readable media, such as on memory 76, a magnetic hard disk, a removable magnetic disk, a magnetic cassette, an optical disk, or from a communication connection, which may also be viewed as a computer readable medium.

Memory 76 includes one or more computer readable media that may be configured to include or includes thereon data or instructions for manipulating data, and may be accessed by processing system 74 through system bus 72. Memory 76 may include, for example, ROM 88, used to permanently store information, and/or RAM 90, used to temporarily store information. ROM 88 may include a basic input/output system (“BIOS”) having one or more routines that are used to establish communication, such as during start-up of computer device 70. RAM 90 may include one or more program modules, such as one or more operating systems, application programs, and/or program data.

One or more mass storage device interfaces 78 may be used to connect one or more mass storage devices 86 to system bus 72. The mass storage devices 86 may be incorporated into or may be peripheral to computer device 70 and allow computer device 70 to retain large amounts of data. Optionally, one or more of the mass storage devices 86 may be removable from computer device 70. Examples of mass storage devices include hard disk drives, magnetic disk drives, tape drives and optical disk drives. A mass storage device 86 may read from and/or write to a magnetic hard disk, a removable magnetic disk, a magnetic cassette, an optical disk, or another computer readable medium. Mass storage devices 86 and their corresponding computer readable media provide nonvolatile storage of data and/or executable instructions that may include one or more program modules such as an operating system, one or more application programs, other program modules, or program data. Such executable instructions are examples of program code means for implementing steps for methods disclosed herein.

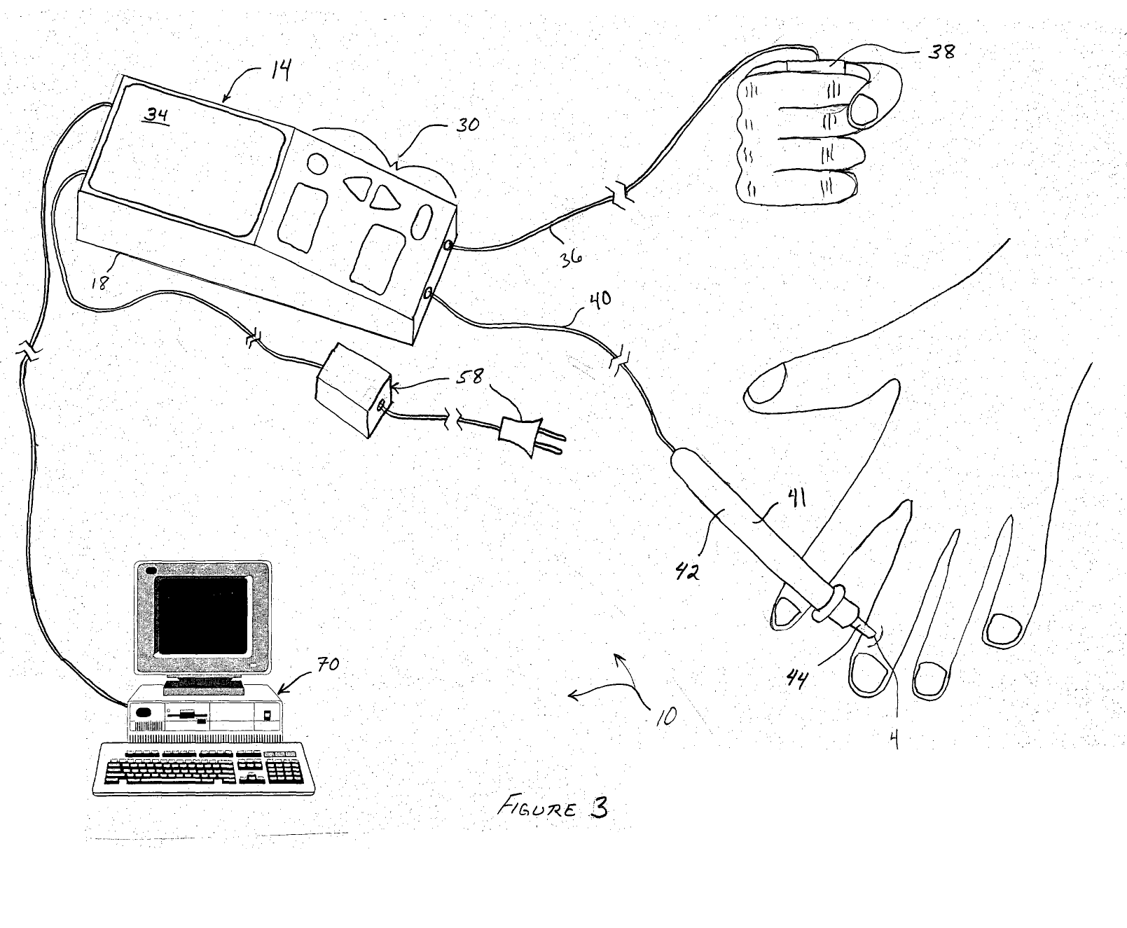

One or more input interfaces 80 may be employed to enable a user to enter data and/or instructions to computer device 70 through one or more corresponding input devices 92. Examples of such input devices 92 include a keyboard and alternate input devices, such as a mouse, trackball, light pen, stylus, or other pointing device, a microphone, a joystick, a game pad, a satellite dish, a scanner, a camcorder, a digital camera, and the like. Similarly, examples of input interfaces 80 that may be used to connect the input devices 92 to the system bus 72 include a serial port, a parallel port, a game port, a universal serial bus (“USB”), a firewire (IEEE 1394), or other suitable interface. Input devices may also comprise a stylus and/or electrode functioning as part of an EDS system or device (see FIG. 2).

One or more output interfaces 82 may be employed to connect one or more corresponding output devices 94 to system bus 72. Examples of output devices include a monitor or display screen, a speaker, a printer, and the like. A particular output device 94 may be integrated with or peripheral to computer device 70. Examples of output interfaces include a video adapter, an audio adapter, a parallel port, and the like.

One or more network interfaces 84 enable computer device 70 to exchange information with one or more other local or remote computer devices, illustrated as computer devices 96, via a network 98 that may include hardwired and/or wireless links. Examples of network interfaces include a network adapter for connection to a local area network (“LAN”) or a modem, wireless link, or other adapter for connection to a wide area network (“WAN”), such as the Internet. The network interface 84 may be incorporated with or peripheral to computer device 70. In a networked system, accessible program modules or portions thereof may be stored in a remote memory storage device. Furthermore, in a networked system computer device 70 may participate in a distributed computing environment, where functions or tasks are performed by a plurality of networked computer devices.

Diagnostic device interface(s) 100 enable computer device 70 to communicate and exchange information with one or more galvanic skin response (GSR) devices or electro-acupuncture by Voll (EAV) devices, and particularly to one or more meridian linking devices 14 as described herein.

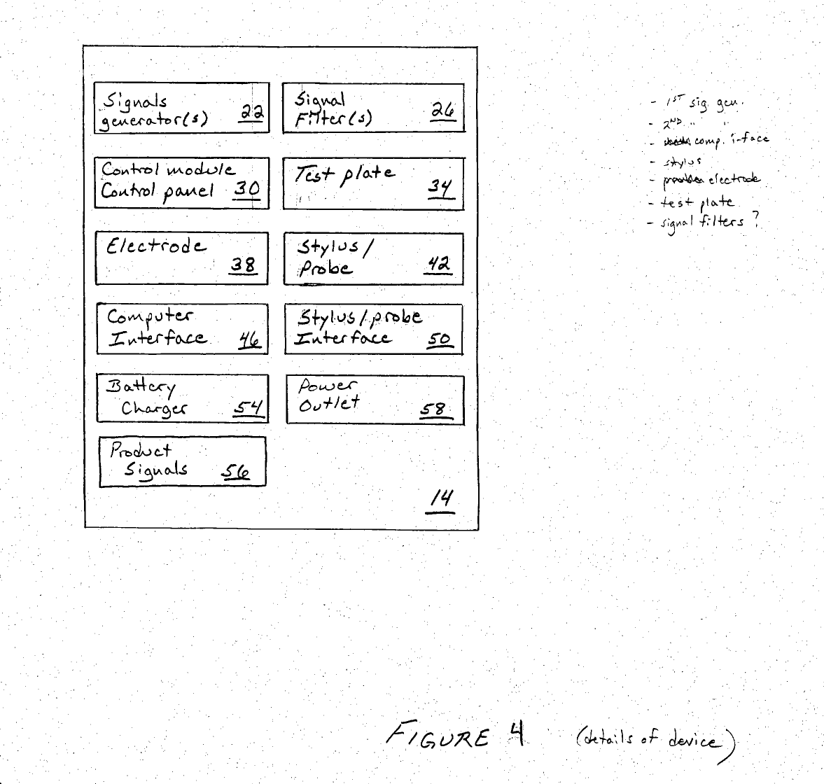

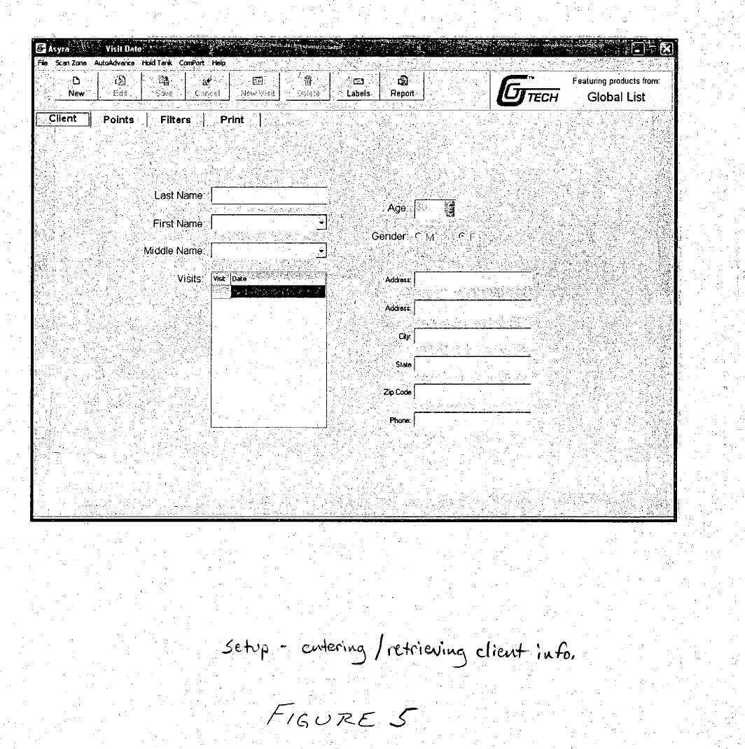

FIG. 2 illustrates an alternative embodiment of meridian linking system 10. The embodiment shown in FIG. 4 is similar to the system shown in FIG. 1, only meridian linking system 10 is a self contained unit with the computer system built into the system allowing it to function as a self-contained computerized unit. FIG. 4 also emphasizes the networking capabilities of the present invention. Meridian linking system 10, as embodied in FIG. 4, comprises networking capabilities. Specifically, FIG. 2 illustrates a network-type configuration, wherein meridian linking system 10 connects to one or more servers 108 via network 98. Other meridian linking systems, shown as systems 112, may also connect to servers 108 via network 98. In this configuration, various remedies databases, electromagnetic signature databases, imprinting databases, or any other databases relating to the diagnosing and treatment of patients using the present invention meridian linking system may be stored on the memory devices of servers 108. Each connected meridian linking system would be allowed to download and upload information as needed, without requiring a great deal of information to be stored directly on the system itself.

### General Discussion of Meridians and Meridian Stress Assessment Testing

The body comprises several meridians that make up an acupuncture meridian system. Meridians are essentially a network of energetic pathways that pass through the organs and tissues of the body, as well as the various systems of the body, such as the nervous system, the circulatory system, the vascular system, etc. Bioelectric energy flows through each meridian and its associated organs and tissues. The Chinese call this energy Qi (pronounced chi).

According to energy medicine theory, every disease state is preceded by and indicated by an energy signal, detectable by various prior art electroacupuncture or similar devices. These devices gather information through the acupuncture meridian system. They can indicate the degree of stress that is affecting an organ and it can monitor the progress of therapy. In addition, it is believed that most conditions can be eliminated or managed by a balance of the meridian with natural remedies, herbal formulas, or nutritional products.

Using these prior art devices, a Meridian Stress Assessment (MSA) test may be performed that helps to assess appropriate ways to achieve an energetic balance in the body, and particularly a balance within the various organs and systems making up the body. Basically, a Meridian Stress Assessment test gives an overview of the status of the meridians in the body. Each meridian is like an electrical circuit. A MSA involves measuring and taking readings of electrical conductivity at various acupoints on the skin using a highly-sensitive ohmmeter to determine the functional status of each meridian in terms of its electric conductance. Electric conductance values are displayed on a computer, which shows the point being tested. The readings indicate whether the meridian (and its associated organ and tissues) is balanced, stressed, or weakened. High values are associated with stress of the related area and low values are associated with weakening of the related area. The electrical current used is very, very small, wherein the only sensation felt by the patient is the stylus touching the skin.

Theoretically, since the body is essentially a communication device, it is constantly receiving and transmitting information coded into energy signals. Changes in the bioenergy field signal the onset of disease before it occurs physically. It is assumed that symptoms of medical conditions in the body have their roots in the energetic system. Thus, if energetic imbalances are determined and substances are found that balance those areas, the body's own systems can be stimulated to resolve the symptoms.

If stress values are above or below the equilibrium range, the test allows consideration of a wide range of possibilities that might help the meridian regain balance. As such, after the status of meridians is determined, various herbal, homeopathic, and nutritional items can be evaluated to see what effect they might have on the stressed or weakened physiological areas, or which might balance any weakened and/or stressed meridian points. For example, over 40,000 different herbal, homeopathic, and nutritional items are available for consideration. And, since changes in matter are usually preceded by changes in electrical conduction, it is simple to assess circuitry.

Overall, a Meridian Stress Assessment provides a completely non-invasive method for gaining information about the condition of the body and its organs and/or systems. The primary objective of the Meridian Stress Assessment is to show patterns of stress and to provide feedback to help restore stressed and/or weakened meridians to an appropriate balance. It should be noted that the term MSA is merely an exemplary term of art and should not be construed as limiting in any way.

As will be shown below, current MSA practice, and other similar practices, are limited in both function and ability. As such, the present invention advances such practices and the equipment or devices used to perform diagnosing and treatment of bodily maladies via the meridians in the body.

### Present Invention Meridian Linking Diagnostic Device and System

The present invention offers a complete paradigm shift in alternative medicine treatments, and particularly in alternative medicine techniques used for treating maladies of a patient through analysis and testing of the meridian in the patient using one or more meridian testing devices or systems (e.g., Electroacupuncture by Voll (EAV), and/or Galvanic Skin Response (GSR) devices or systems). The present invention describes a unique GSR or EAV system and method that links the meridians of the body into an interconnected network. This network further links all of the internal body systems and organs to the one or more data access or reference points existing on an area of the body, namely the hands. The present invention also functions to stabilize one or more data access points as reference points, wherein diagnosing and treatment of various manifested and latent maladies (e.g. diseases, pains, or other illnesses or discomforts) in a patient may be performed through a single reference point. The present invention further functions to stress the body using a set of frequencies, wherein the stable reference points are monitored for change. Still further, the present invention functions to associate remedies with tested maladies, and to automatically load these remedies into the system for treatment of the patient.

Stated another way, the present invention strengthens and stabilizes the links between the meridians in the body, thus creating a total systemic network that is linked to one or more reference points. The linking of the body's meridians into an interconnected network allows the user to test the entire body using only one of the identified stable reference points. The present invention also utilizes “Ting points,” which are more energetically active and easier to locate. Once the reference points are established the present invention outputs a filter that stresses the body. As the body is stressed, the reference point resistance value is measured or read to see if the network was affected. If so, rather if the point registers as unstable, then this indicates a problem and the system moves to the remedy finding function, wherein one or more remedies, corresponding to the particular malady being tested for, are automatically loaded into the system for further testing to ascertain their effect on the discovered malady. Each of these functions, namely the meridian linking function, the point stabilization function, the stressing function, and the remedy finding function, are each unique to the present invention and are not found in the prior art.

This paradigm shift of concepts and unique GSR or EAV system is accomplished through use of a computerized GSR or EAV meridian linking diagnostic and treatment system (hereinafter “meridian linking system”), such as the Asyra™ meridian linking device and system produced and offered by GTech, L.L.C. of Saratoga Springs, Utah 84043.

With reference to FIG. 3, shown is meridian linking system 10 comprising generally a meridian linking device 14 and computer or computer system 70. Meridian linking device 14 further comprises a primary base unit 18 that supports all of the internal components within meridian linking device 14, a power outlet 58, a stylus or probe 42, an electrode 44, a control panel or control module 30 for controlling the functions of meridian linking device 14, and a test plate 34. Preferably, control module 30 is in the form of a touch-pad with buttons thereon for controlling functions, such as the power on/off, the volume of the relayed audio from taken readings or measurements, and the calibration of meridian linking device 14 (although device 14 is preferably a self-calibrating device with optional manual override). Of course, other controls may be incorporated for additional features of meridian linking device 14.

Test plate 34 facilitates or allows for testing of products/remedies that are not initially or previously stored or contained in the system database. Test plate 34 and the test plate function are each described in greater detail below.

Electrically coupled to primary base unit 18 of meridian linking device 14 are electrode 38 and stylus or probe 42. Each of these function in a similar manner as known in the art. Essentially, there are two cables coming out of the meridian linking device 14, a negative lead 36, and a positive lead 40. Negative lead 36 is attached to electrode 38, and positive lead 40 is attached to stylus 42 having an electrode tip 44. The technician or clinician holds stylus 42 by the insulated handle 41 and presses tip 44 against one of the patient's acupuncture points 4. The patient holds electrode 38, which is a hand electrode, in their free hand. During the measurement the patient and meridian linking device 14 form a closed circuit, allowing energy and information to flow from meridian linking device 14 to stylus 42, through the patient to electrode 44, and back to meridian linking device 14. The EAV or GSR reading is a measurement of how much energy makes it through the circuit (the lower the resistance the higher the reading). Specifically, the reading is a measurement of inductance.

With reference to FIG. 4, meridian linking device 14 comprises various internal components. Specifically, meridian linking device 14 comprises a signal generator 22 that generates and broadcasts the filters or signal frequencies utilized by meridian linking system 10. When the system is activated, signal generator 22 outputs two permanent filters used for strengthening and linking the meridians in the body and for establishing and stabilizing the several reference points, namely the meridian linkage/coupling filter and the point stabilization filter, respectively. These two permanent filters are continuously broadcast in an alternating manner while meridian linking system 10 is active. The linking of the meridians in the body is unique to the present invention and is not found in prior art devices. The present invention recognizes the meridians as a complete interconnected network and outputs the necessary signal to link these meridians to the one or more stable reference points, wherein any one of the points may be used to test the internal functions of the entire body.

Signal generator 22 also functions to output the several pre-determined or pre-identified filters designed to stress the body and diagnose both manifested and latent maladies in the body. These pre-determined filters are also broadcast in an alternating manner with both the meridian linkage/coupling and point stabilization filters. These filters may be amplified for additional benefit, as will be explained below.

Meridian linking device 14 comprises other hardware components, such as signal filters 26 (e.g., amplification filters), control module or panel 30, test plate 34, electrode 38, stylus or probe 42, computer interface 46, stylus/probe interface 50, battery charger 54, product signals 56, power outlet 58.

Meridian linking system 10 further comprises a computer device and/or computer system 70 that exchanges information with meridian linking device 14 via a communication link (e.g., USB, wireless, Serial, etc.), that stores the several predetermined filters in a database, provides a unique user interface, and that otherwise controls the functions of meridian linking device 14 using several computer software modules or Functions that are detailed below. Computer system 70 facilitates or allows meridian linking system 10 to operate and function in its intended manner described below. The particulars pertaining to computer device and/or computer system 70, and particularly its hardware and setup details, utilized in the present invention are described above in the Section entitled, “Exemplary Computerized Operating Environment section.”

As stated, computer or computer system 70 comprises or is capable of executing thereon, several software modules or Functions comprising code embedded on a computer-readable medium that is used for controlling meridian linking system 10, and particularly meridian linking device 14 and computer system 70, to perform as intended and described herein. Each of these computer software Functions are described in greater detail below.

### Client and System Setup

Turning now to FIG. 5, meridian linking system 10 comprises various client or patient, clinician or practitioner, and system setup modes. For example, the user can add or change the clinic information, perform system calibration, create and modify patient information, access system databases, and any other type of setup procedures required or desired to be performed by the user. FIG. 5 depicts a screen-shot of an exemplary client/patient setup page. From this page the user perform several operations, such as entering information for a new patient (e.g., name, age, gender, address, results of previous tests, etc.), selecting a new visit for a previously tested patient, creating and editing or modifying patient information, retrieving patient data from a previous visit to review and/or print, and/or deleting a patient record or a specific patient visit from their record. Any client setup information may be saved in the system.

To begin a testing session, a new or previously tested patient must be identified. If the patient is a new patient, the appropriate information is entered into the system to allow the system to prepare for the initial testing of that patient. If the patient has already been tested and it undergoing a subsequent test, the patient is selected from the database of patients presented to the user preferably in a list form or based on a searching function, wherein the patient's information is loaded into the system in preparation for the test.

### Meridian Linking and Data Access Point Stabilizing Functions

One of the primary features of the present invention is its Meridian Linking and Point Stabilization Function. The meridian linking system technology makes use of the body's natural electronic properties, namely capacitance, inductance, resistance and conductance. Viewing the body's natural electronic components as a basic function of the body, and reducing these components down to their electronic equivalent circuits provides an energetic pattern of the body's functional status. The meridian linking system is used to enhance, strengthen, stabilize, and measure the body's energetic status and response to external stimulus. First, a look at the body's electronic equivalent circuit will aid in an understanding of the role these circuits play.

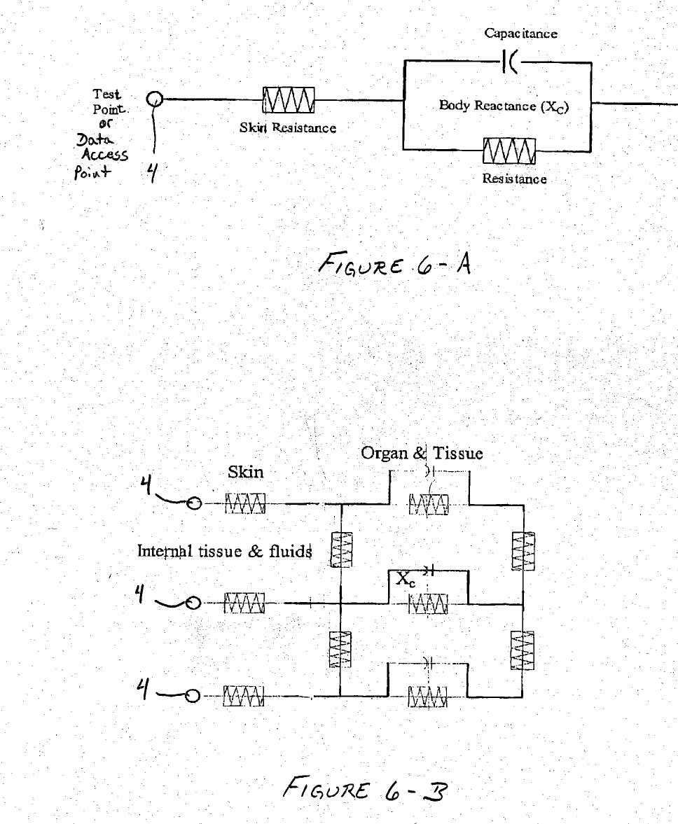

With reference to FIG. 6-A, the equivalent circuit for a single function in the body is illustrated. While the body has (in greater or less degree) all four of the components listed above, inductance is almost negligible. And, since resistance and conductance are inversely related (i.e., they are the reciprocal of one another) it can be concluded that two appropriate units of measure comprise the resistance and capacitance in the body. These two components effectively provide for the basic electronic equivalent circuit of the body.

FIG. 6-A generally illustrates the inner workings of the body's electronic function. The circuit in FIG. 6-A forms a component that is frequency responsive and is known as capacitive reactance (X_{C}). The nature of capacitive reactance is that, as the frequency of a given signal is increased, the resistance or the “Z” of the reactance circuit decreases. This relationship is described in the following formula:

Z\sqrt{R²+(X_{L}−X_{C})²}

where R=E/I (Resistance), X_{L}=2πFL (Inductive Reactance), and X_{C}=½π FC (Capacitive Reactance).

The Meridian linking system technology of the present invention advances prior art GSR and EAV devices and systems and alters conventional MSA thinking by recognizing that the body's circuits are not singular in nature, but are part of a network of interconnected circuits similar to a computer network, wherein information resides in a number of locations and can be accessed from a local or single data access point. Unlike prior art systems that treat each meridian independently from one another and that look or test each meridian individually, the present invention meridian linking system effectively links all the meridians in the body together and also to one or more stable reference points so that only a single point is needed to perform the diagnosing and treatment functions. Also, unlike prior art systems that only look for and test or measure the energetic balance of each meridian, the present invention meridian linking system actually stresses the internal functions of the body and measures the response of the entire meridian network to that stress. The body's equivalent circuit for the above described network is illustrated in FIG. 6-B.

It can be seen from this equivalent circuit that a given frequency sent or broadcast from a single data access point to the systems in the body would have the effect of changing the capacitive reactance of organ tissue sets existing within the body's interconnected network. It is also understood that all circuits will fall into a specific resistive range because of the frequency response of the individual circuit. It is this resistive range that is preferably measured by the meridian linking system of the present invention to determine and establish one or more reference points and to diagnose and treat any maladies in the patient.

As explained above, the body comprises several data access points. Many of these data access points are located on the hands. The present invention contemplates utilizing these data access points as reference points for testing purposes similar to prior art EAV or GSR devices. In a preferred embodiment, the meridian linking system utilizes the Ting points as they are more energetically active and easier to locate. However, one of the primary functions of the present invention is to locate and define data access points that are “stable” as these “stable” data access points are identified and stored as reference points for testing purposes. A “stable” data access point is defined herein as a data access point that comprises a resistance measurement that falls within a pre-determined or pre-identified resistance range, as set or established by the user prior to testing. The resistance range or reference point parameters with their upper and lower values is/are referred to herein as the “scan zone.” In one exemplary embodiment, a preferred range of the scan zone comprises an upper resistance value of 55 and a lower resistance value of 45. Thus, if a measured data access point falls between 45 and 55, the data access point is denoted as a “stable” point and is used as a reference point for the entire network. If the measured data access point falls without this zone it is disregarded.

To locate and identify a “stable” data access point, several data points are tested using the meridian linking device described above. The probe or stylus electrically coupled to the device is brought into contact with the skin and caused to probe along the surface of the skin until a data access point is located. Identifying the location of data access points is common in the industry and not specifically described herein. However, identifying and maintaining “stable” data access points as reference points is unique to the present invention.

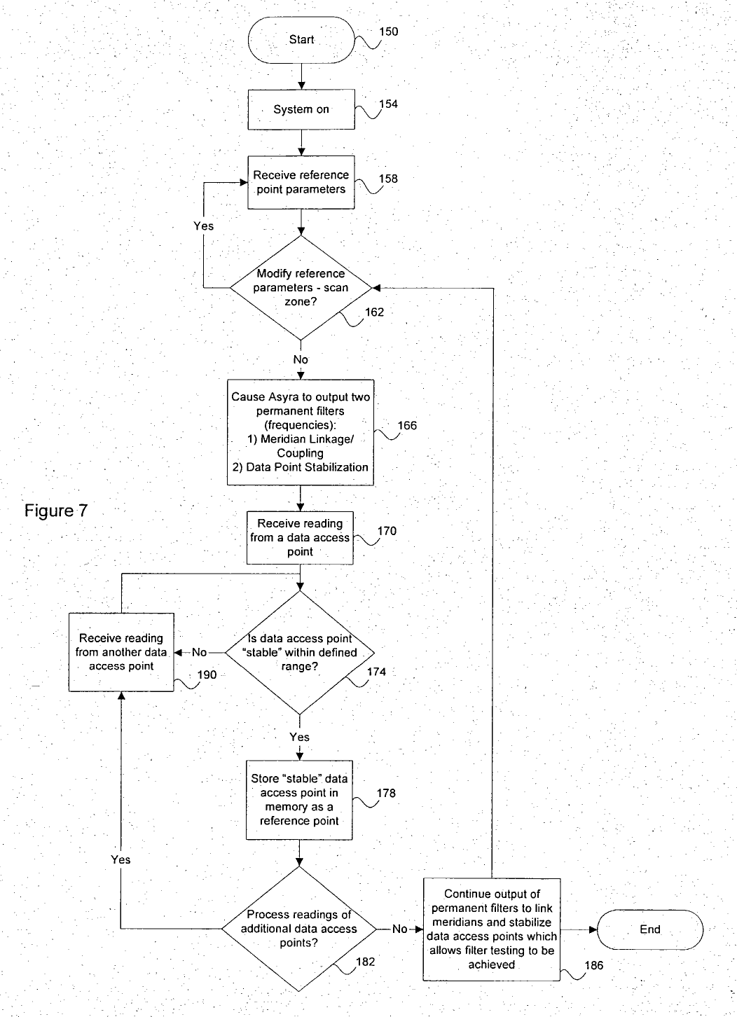

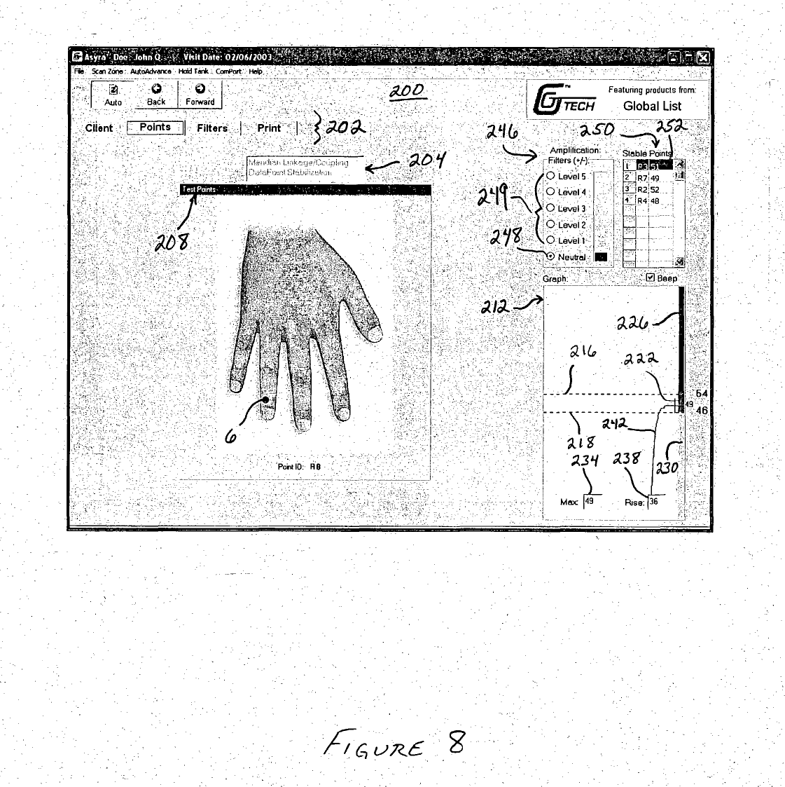

With reference to FIG. 7, shown is a flow chart of the Meridian Linking and Data Access Point Stabilizing Functions, as performed by the computer system in combination with the meridian linking device, according to one exemplary embodiment of the present invention that is used to identify and maintain a series of stable reference points. To begin the process, step 150, the meridian linking system is powered and activated, or turned to its “on” position—step 154. After the system is activated, the computer receives and processes the initial scan zone parameters approved by the user (e.g. either set by the user or the manufacture) that define the upper and lower resistance values that a data access point must fall within to be considered “stable”—step 158. As stated above, it is this scan zone or resistance range that dictates or defines whether a data access point is “stable,” such that it may be stored and used as a reference point. In one embodiment, when the system is activated, the scan zone is set to a default setting previously defined by the user or set by the manufacture. In another embodiment, the user sets the scan zone each time the system is activated. In any event, the scan zone may be modified as needed—step 162. Or, in other words, the upper and lower values or parameters of the scan zone can be modified at any time by the user as needed or desired. Modifying the scan zone allows the user to perform much more scrutinizing diagnosis and treatment procedures. In addition, modifying the scan zone simultaneously affects the parameters used for stable points, filter testing, and the remedy/product scan.

If no modifications to the scan zone are performed, the system proceeds to step 166. In step 166, the computer causes the meridian linking device to output two permanent filters or frequencies—a meridian linkage/coupling filter and a data access point stabilization filter—step 166. In regards to identification and maintenance of stable reference points, using a signal generator, the computer causes the meridian linking device to output a low voltage and low current data access point stabilization filter or frequency that measures or reads the resistance or resistance range of that particular data access point based on a response of the organ tissue set to the output frequency. In step 170, the computer receives and processes the measurement or reading of a particular data access point. Taking a measurement of a data access point is a critical process that must be performed properly to obtain an accurate reading. For example, improper measuring can cause inflammation in the skin, thus resulting in an false low value.

The processing step of 170 further comprises the computer determining whether the resistance measurement taken from the data access point falls within scan zone. In the event that the resistance of an organ tissue set, because of the frequency response of the organ tissue set at that particular data access point, falls into the pre-identified or pre-defined range of the scan zone, then that data access point is defined as “stable” and is be stored in the meridian linking system stable points list as a reference point—step 178. A reference point is thus defined herein as a stable data access point that is used for the present invention testing and treatment procedures. If the point falls without the scan zone, that particular point is disregarded and the user moves on to another data access point, step 190, and repeats the process. It should be noted that not all of the data access points can be stabilized. Sometimes the influence of an organ is so strong that it disrupts the ability of the permanent filters to stabilize a specific data access point. Thus, it is advantageous to identify several data access points that are stable. As such, the above process may be repeated for any number of data access points until it is determined that a sufficient number of stable reference points have been identified and stored in the system memory—step 182. Preferably, three to five reference points are needed to perform the filter testing function described herein. Of course, any number of reference points may be obtained until all data access points are exhausted or until the stable points list is full.

The meridian linkage/coupling filter and the data access point stabilization filter output by the meridian linking system are each permanent filters, meaning that their given frequency is continuously broadcast or output throughout the entire testing and treatment session of each patient—step 186. In other words, the meridian linking system continues to output each of these signals for the purpose of linking the meridians in the body and to stabilize the reference points that allow filter testing to be performed. Because the signals are permanent, each meridian is linked in an interconnected network that itself is linked to each reference point, and the “stable” status or integrity of each of the reference points is maintained.

In another embodiment, the meridian linking system comprises an auto advance feature, wherein upon measurement of a data access point, the computer automatically advances to the next data access point for measurement. Only those data access points that measure within the scan zone are stored within the system memory while those that fall without the range are disregarded.

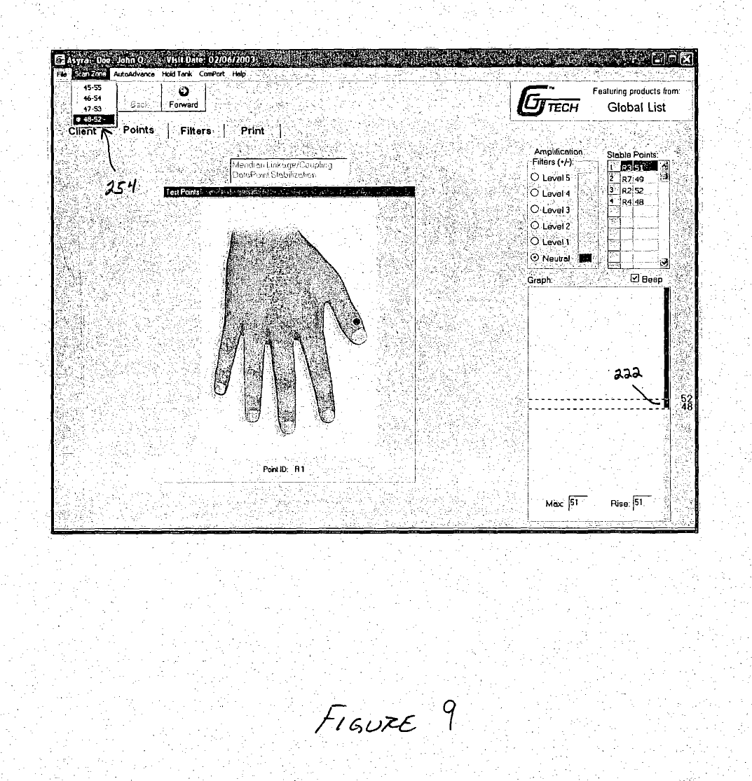

FIG. 8 illustrates a screen-shot of an exemplary graphical user operating interface comprising many of the features of the present invention. Specifically, FIG. 8 illustrates a current operating environment 200. The present invention comprises several operating environments depending upon or corresponding to the several functions available with the meridian linking system. In the exemplary embodiment shown in FIG. 8, the current operating environment 200 comprises a data access point location field 208, wherein the location of the several data access points 6 on the hands is displayed. As discussed above, data access points are tested for the purpose of locating and establishing one or more reference points. These data access points are commonly known in the art and their general vicinity may be displayed and programmed or stored in the system. Of course, if other parts of the body are used for testing, then the corresponding location of the data access points for the part of the body being tested will be displayed within data access point location field 208.

Other operating environments are provided, as indicated by the several tabs existing within environment/function control field 202. The user may selectively toggle between these operating environments to activate the various functions of the meridian linking system. Using the interface shown in FIG. 8, all the user has to do to switch between the different operating environments, and therefore the different functions of the meridian linking system, is select the appropriate tab.

FIG. 8 also illustrates permanent filter field 204 that displays which filters are currently being broadcast. In the embodiment shown, permanent filter field 204 is displaying or indicating that both the meridian linkage/coupling filter and the data access point stabilization filter are currently being broadcast to the patient.

Also shown is point status indicator field 212 that comprises a graphical representation or readout 242 of each data access point being tested. Graphical readout 246 comprises a rise 238 and a maximum value 234 that portray the properties of the tested data access point. Point status indicator field 212 further comprises an upper parameter display 216, a lower parameter display 218, and a stable point range display 222, otherwise known as the scan zone. Upper parameter display 216, shown having a resistance value of 56, corresponds to an upper resistance parameter or value selected or authorized by the user that defines the highest resistance value a measured point may measure to still be considered a “stable” point. If the reading or measurement of a data access point is above this upper parameter then it is considered unstable and disregarded and its value will be indicated somewhere in upper imbalance area 226 depending upon its value. Lower parameter display 218, shown having a resistance value of 46, corresponds to a lower resistance parameter or value selected or authorized by the user that defines the lowest resistance value a measured point may measure to still be considered a “stable” point. If the reading or measurement of a data access point is below this lower parameter, then it is considered unstable and disregarded and its value will be indicated somewhere in lower imbalance area 230. Both upper and lower imbalance areas 226 and 230 are without scan zone 222 and signal that the data access point is unstable. In addition, returned readings that fall outside the scan zone and within these imbalance areas signal an imbalance in the organ or system being tested. In FIG. 8, the measured value reads 49, meaning the reading from the data access point falls within the scan zone and is stable.

Each of upper and lower resistance parameters, thus defining the scan zone 222, may be modified as desired or required by the user. Any modification of these values will be reflected within point status indicator field, and particularly upper and lower parameter displays 216 and 218, respectively. An example of a modified scan zone 222 is illustrated in FIG. 9, wherein an upper resistance parameter comprises a value of 52 and a lower resistance parameter comprises a value of 48. These values can be manually entered into the system, or they can be selected from a list 254 of pre-determined scan zone parameters. This scan zone has a range that is much narrower than the one illustrated in FIG. 8, thus facilitating more scrutinized and precise stable point analysis in each of the stable reference point establishment, diagnosis, and treatment processes performed by the meridian linking system of the present invention. Changing the value of the scan zone simultaneously affects the parameters used for stable points testing, filter testing, and remedy scan.

FIG. 8 further illustrates amplification field 246. Amplification field 246 functions to display the current 248 and available 249 filter amplification levels of the filter being broadcast. If the user desires or is required to increase or decrease the amplification level of the broadcasted filter, the user simply enters the appropriate environment displaying amplification field 246 and selects the proper amplification level from the list of available levels 249. The amplification function or feature of the present invention is discussed in greater detail below.

Finally, FIG. 8 illustrates stable points field 250 that indicates or displays to the user the list of available stable reference points established during the stable reference point setup procedure. Each stable reference point is identified by an identifier corresponding to its location and tested resistance value. Also within stable points field 250 is the display 252 of the current selected stable reference point being utilized by the meridian linking system. At any time, the user may toggle back and forth between these points as necessary. For example, if one point suddenly becomes unstable as a result of user error, system malfunction, or change in physiological conditions, the user may switch to another referent point to complete the current procedure. Each time a data access point is identified as being “stable,” it's identifier is stored in the stable points list as a reference point.

### Filter Testing Function

In addition to the method and system for establishing several, or at least one, stable reference point(s) and for linking the meridians in the body together and to the various stable reference points, the present invention further features a computer software Filter Testing Function that operates or functions with the meridian linking and point stabilization features to diagnose and treat both manifested and latent maladies within the body. The specific function of the Filter Testing Function is to induce a stress within the body by outputting a customized pre-determined filter corresponding to a specific malady in order to identify and diagnose both existing and latent maladies existing within the body. Stated differently, each customized filter comprises an assigned malady-specific frequency designed to stress the body with the specific malady to determine if an imbalance is created at the stable point. If an imbalance is created, the patient may suffer from the specific malady. For example, if a patient suffered from allergies, but was unaware of what was causing the allergies, one or more customized filters corresponding or specific to various substances known to cause allergies would be broadcast to the individual, thus stressing the body with each substance. This would allow the clinician to determine which substance the causing the allergies in the patient and would allow the clinician to formulate a proper treatment.

Stressing the body is unique to the present invention and not found in prior art EDS systems. Although some systems are capable of diagnosing manifested maladies, the ability to diagnose and treat latent maladies is unique to the present invention and provides significant advantages over the prior art. Several of these advantages are specifically recited herein, while others will be apparent from the detailed description of the system and method as provided below.

Several pre-determined customized filters may be loaded and stored in the memory databases of the computer system. Each of these customized filters represents or corresponds to one or a combination of maladies that may develop within or infect a patient. As such, the filter database is typically quite large. The customized filters each comprise a frequency that is specific to that filter. The customized filters are designed to stress the body with certain conditions. If the body maintains its homeostasis in the presence of the filter, as indicated by a stable point measurement, then there is no underlying problem or malady in the body for which that particular filter is associated. If however, if any energetic component, either cellular, tissue, organ, or system, responds adversely to the filter, as indicated by an unstable point measurement, then there exists either a manifested or latent malady that corresponds to the customized filter. Each filter may also be named or otherwise coded for purposes of storage and organization within the database, wherein they may later be retrieved by the appropriate computer software function.

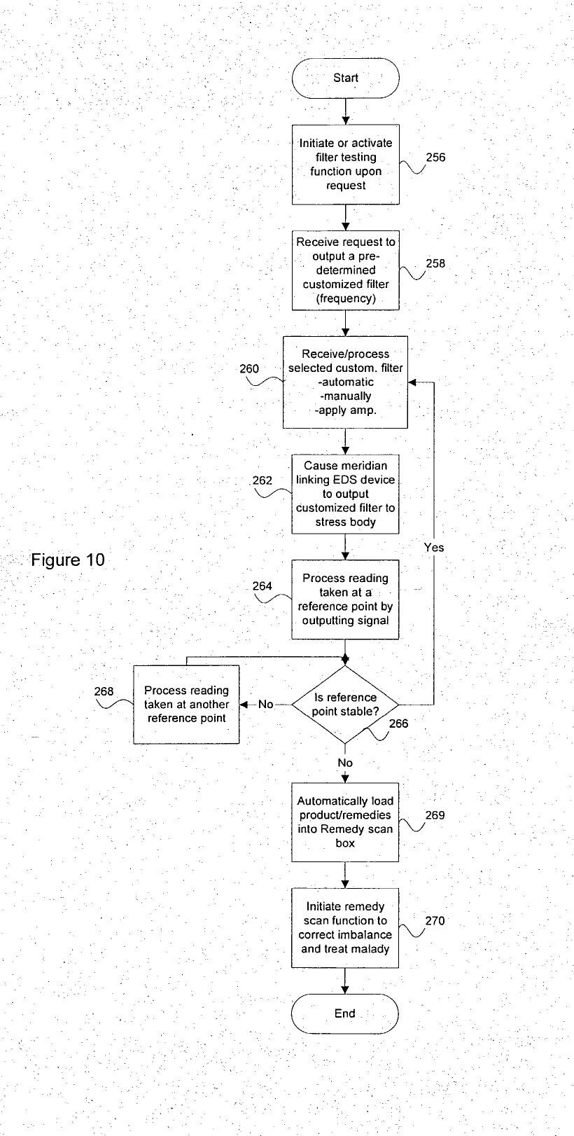

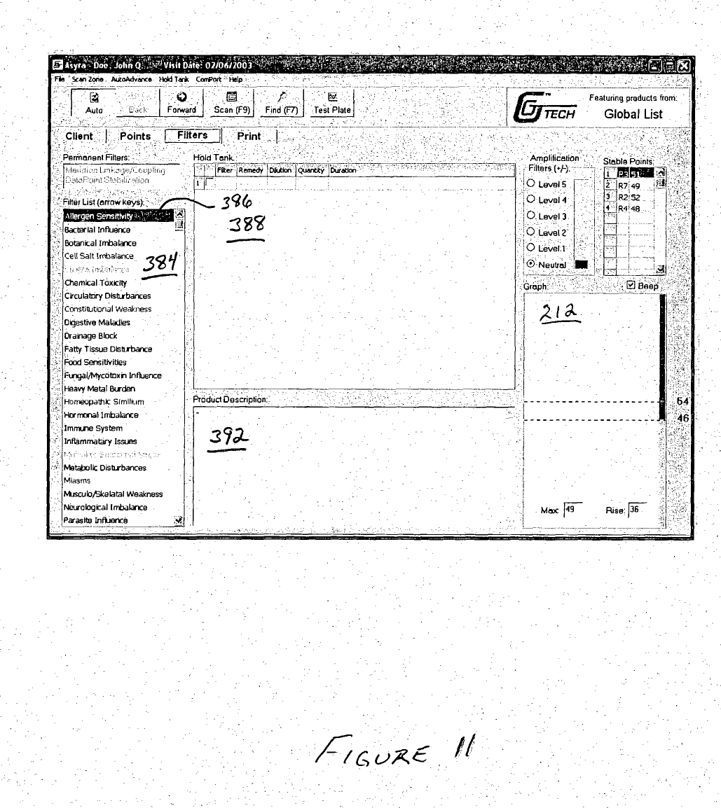

With reference to FIG. 10, once the computer system activates or initiates the Filter Testing Function as requested or directed by the user—step 256, the computer receives a request from the user for the computer to output or broadcast a pre-determined customized filter—step 258. As mentioned, filters are designed to stress the body with certain conditions. If the body can maintain homeostasis, the reading will stay within the scan zone. However, if any internal bodily physiology responds adversely, thus indicating an existing or underlying or latent malady, an imbalanced reading (either above or below the scan zone), will result.

Once the desired filter is selected by the user, the computer processes this request, step 260, which includes retrieving the filter from the filter database, and causes the meridian linking device, via the Filter Testing Function, to output or broadcast the selected customized filter for the purpose of stressing the body—step 262. As the filter is being broadcast, the user then takes a reading at one of the stable reference points via the permanent point stabilizing frequency, wherein the reading is sent back to the computer for processing—step 264. If any of the organ/tissue sets are not affected, the reference point will measure within the scan zone, which indicates that the particular customized filter chosen was not associated with any malady in the patient's body. Thus, the user selects another customized filter and the computer processes this request, step 260, to cause the meridian linking system to output the newly selected filter. This process can be repeated as many times as there are customized filters.

However, if any of the organ/tissue sets are affected it will change the overall resistance of the body network and the meridian linking system will detect and measure the change at the measured stable reference point(s)—step 266. To make sure the resistance of the body did actually change and not the stability of the reference point, a second measurement can be taken at another reference point, wherein the computer processes this reading and returns the measured resistance value—step 268. A reading outside of the scan zone indicates an imbalance in the meridian network, and thus the organ/tissue set, and that the body's energetic balance can be restored by the use of the specific filter that was used to cause the network to change. Once an imbalance is discovered, the computer automatically loads the products/remedies in the scan box—step 269. At this time, the computer also initiates or activates, either automatically or at the request of the user, the Remedy Scan Function of the meridian linking system—step 270.

In regards to step 260, there are several ways that the user can sort through the available filters from the customized filter database to see which ones maintain homeostasis or which ones induce an adverse affect. In one exemplary embodiment, the user can test through all of the filters automatically. In another embodiment, the user can manually select the desired filters and test one or more of them individually. Still in another embodiment, the user can apply the amplification mode that increases the intensity of the filter, thus allowing the user to gather more scrutinizing and precise data potentially uncovering latent or subtly manifested maladies.

To manually test through the filters, each desired filter is selected from the filter list. Using one of the stable reference points from the list of stable reference points, a reading may then be taken to determine if that particular filter causes an imbalance in the linked meridian network. If the filter does not cause an imbalance, it may be concluded that the patient is free of the malady corresponding to the filter and another filter is selected from the filter list for testing.

If the filter does induce or cause an imbalance, it may be concluded that the patient is afflicted with the malady corresponding to the broadcast filter. Upon receiving an imbalance reading, the meridian linking system will automatically load the products/remedies to scan to find the most appropriate product/remedy to restore balance or homeostasis in the patient. Once the most effective or proper product/remedy has been identified for the desired filter, it is caused to be stored in the hold tank. This process may be repeated as often as necessary or for as many filters as are desired to be tested to properly treat the patient.

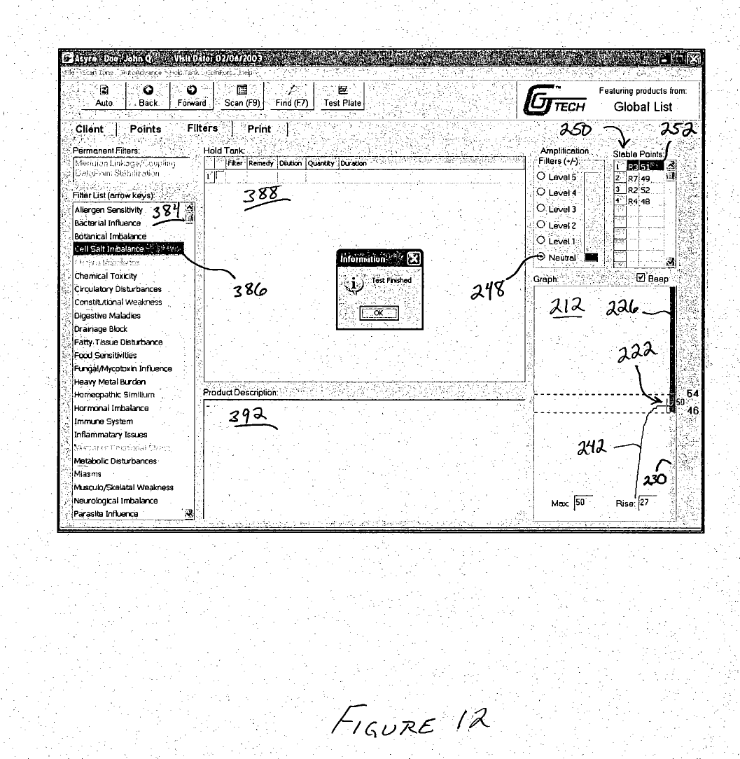

FIG. 11 illustrates a screen-shot of an exemplary filter testing environment comprising a filter list 384, a holding tank display 388 showing which products/remedies and filters are stored therein, a product description field that shows a detailed description of any products/remedies in the holding tank, and point status indicating field 212. From within filter list 384, a filter 386 may be selected for testing.

Similar to FIG. 11, FIG. 12 illustrates a screen-shot of an exemplary filter testing environment, wherein, upon testing, the selected filter 386 returned a measured resistance value of 50 as indicated by graphical reading indicator 242 in point status indicator field 212, which is within scan zone 222, thus indicating the patient is free of the malady corresponding to selected filter 386. As such, testing of selected filter 386 is finished as indicated. FIG. 12 also illustrates selected stable reference point 252 from stable reference points list or field 250 and an amplification 248 of neutral.

To automatically test through all of the filters, an auto advance mode is activated in meridian linking system 10. In this mode, the computer causes or directs the system to automatically test through each of the filters in the filter list. Beginning with the first filter in the list, a reading is taken using one of the stable reference points. If the filter does not cause an imbalance, the computer automatically advances to the next filter in the list. If, however, the filter does induce an imbalance, the system computer, via the Filter Testing Function, will automatically cause the corresponding products/remedies to load, wherein these may be analyzed and tested to find the most effective and appropriate treatment. This process is repeated until all of the customized filters in the list have been exhausted, or at least all those desired or chosen for testing. Again, once the most effective and/or appropriate product/remedy is discovered, it is placed in the hold tank.

In each of the manual or automatic filter testing modes, amplification of any selected filter may be used to improve the accuracy of any diagnosis and to reveal latent or subtly manifested maladies as amplification increases the filter strength so that an even greater stress is induced within the patient depending upon the amplification level chosen.

Each filter in the filter list may also comprise an identifier, such as appearing as different colors in the user interface, to classify or categorize the filters into groups or subgroups.

### Remedy Scan/Finding Function

The present invention further features a computer software Remedy Scan/Finding Function (hereinafter “Remedy Scan Function”). The Remedy Scan Function is activated after the Filter Testing Function and associated diagnosing process has been completed and one or more customized filters are discovered to create an imbalance at one or more of the stable reference points. The present invention comprises a product/remedy database comprising a plurality of product/remedy filters that are designed to balance against each customized filter capable of being broadcast by the meridian linking system. Product/remedy filters (products/remedies) are preferably automatically loaded, but these can be manually or selectively loaded for the purpose of providing potential treatment solutions for the discovered imbalance. Each product/remedy filter is testing to determine if it, or a combination of product/remedy filters, can restore homeostasis in the patient. Specifically, in a preferred exemplary embodiment, the meridian linking system comprises executable computer code that directs the computer to automatically load one or a plurality of products/remedies for further testing.

Once one or more products/remedies are loaded, the Remedy Scan Function controls or causes the computer to go through an elimination process to determine which of the loaded products/remedies are the most effective and/or the most appropriate for the patient and his or her discovered condition. The Remedy Scan Function focuses on narrowing a large list of potential treatment remedies down to the single, most effective or appropriate remedy. A single product/remedy is preferred, but there may be two or more remedies that are discovered to provide an effective treatment of the malady.

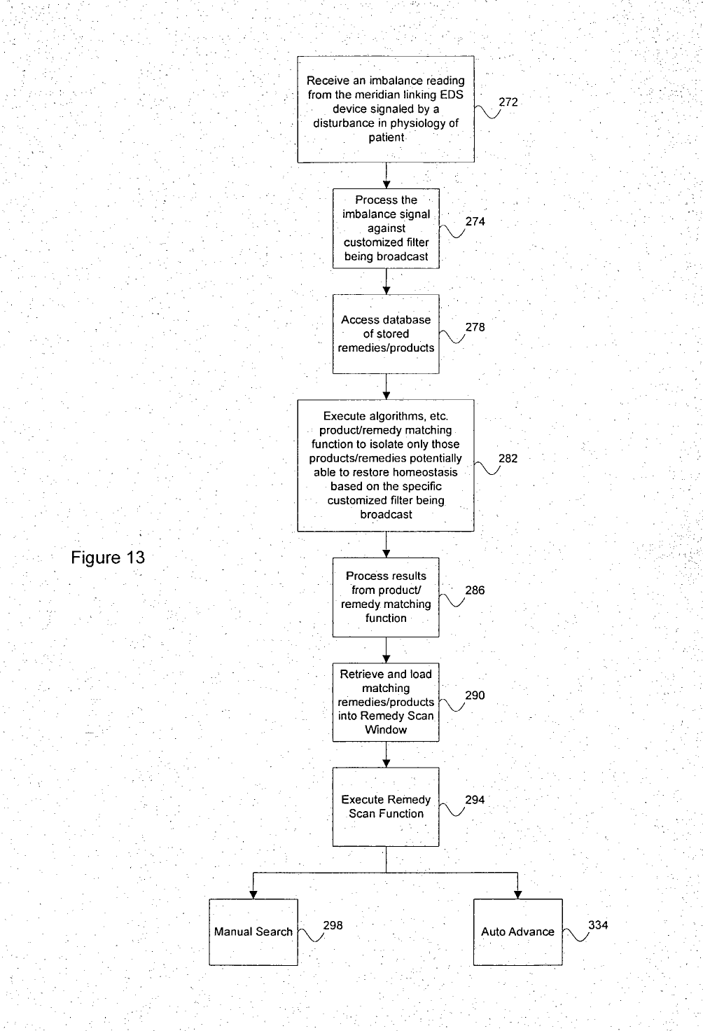

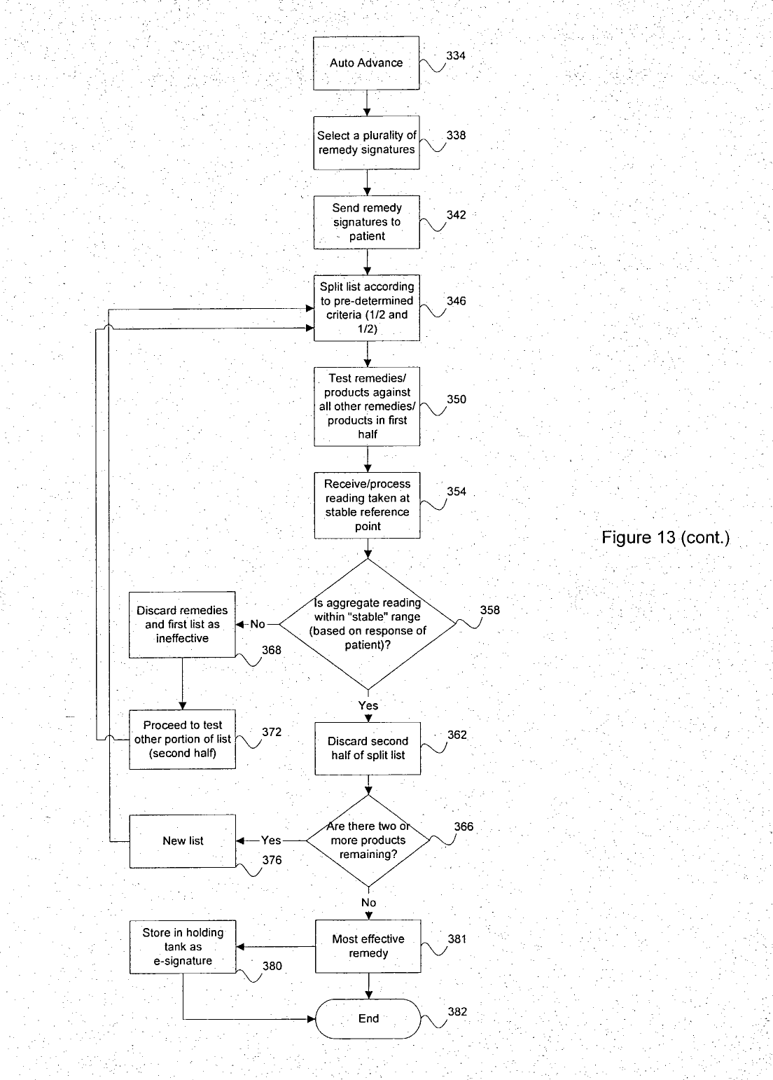

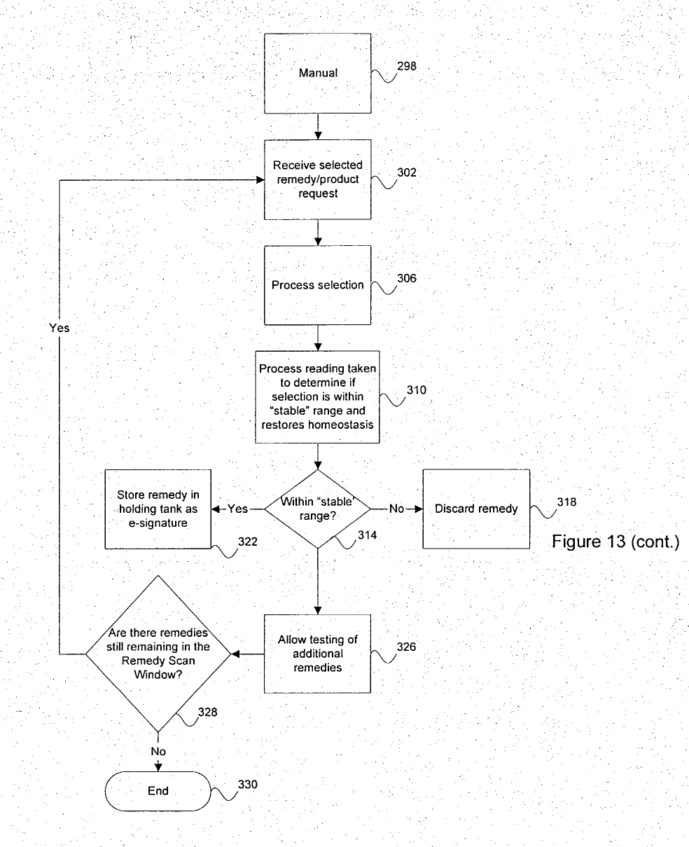

FIG. 13 illustrates a flowchart of the computerized Remedy Scan Function, including steps leading up to activation of the Remedy Scan Function. First, the computer receives and processes an imbalance reading as taken at a stable point by the meridian linking device during the Filter Testing Function, wherein the imbalance reading signals a disturbance in the physiology of the patient or a malady, either manifested or latent—step 272. The computer then processes the imbalance signal against the customized filter being broadcast, step 274, and accesses a database of stored products/remedies, step 278. The computer then executes one or more algorithms, etc. in a Product/remedy Matching Function designed to isolate only those products/remedies potentially able to restore homeostasis based on the specific customized filter being broadcast—step 282. The computer then processes the results obtained from the Product/remedy Matching Function, step 286, and then retrieves and loads the matching products/remedies into a remedy scan window. From here, the Remedy Scan Function is activated or initiated, step 294, either manually by the user, step 298, or automatically, step 334.

Manually activating the Remedy Scan Function, step 298, includes causing the computer to receive and process the product/remedy selected by the user from the list of products/remedies loaded by the computer—steps 302 and 306. As each of the products/remedies are selected by the user, a reading is taken at the stable reference point. The computer processes the readings and whether the reading is within the stable scan zone—step 310. If the reading is within the stable scan zone, the product/remedy is effective against the particular customized filter being broadcast that created an imbalance in the patient. In other words, if the reading is within the scan zone, that particular product/remedy is capable of restoring homeostasis to the patient and will be effective at treating the malady of the patient diagnosed by the customized filter. Thus, if the reading is within the scan zone, the computer causes the system to store the product/remedy in the hold tank and associate an electromagnetic signature with the product/remedy—step 332, which is a digital representation of the effect of the product/remedy on a biological entity. If the product/remedy does not restore homeostasis or is without the scan zone, that particular product/remedy is discarded as ineffective—step 318. Step 326 allows other product/remedies to be tested. If there are additional loaded product/remedies remaining, each of these can be tested to determine if they restore homeostasis or are ineffective using the procedure described above—step 328. Once all of the remedies have been tested, the Remedy Scan Function is completed and is deactivated—step 330.

Automatically activating the Remedy Scan Function using the auto advance feature, step 334, includes causing the computer to select a plurality of products/remedies from the list loaded by the computer via the Filter Testing Function—step 338. The computer then is caused to send each of these remedy signatures to the patient—step 342. The Remedy Scan Function then causes the computer to split this list according to pre-determined criteria (preferably in halves), step 346, and to test each product/remedy in one segment against all other products/remedies in the other segment of the list—step 350. A reading is then taken at the stable reference point for that particular group and processed by the computer—step 354. The aggregate reading for that group will either fall within the scan zone, thus signaling that homeostasis is restored, or the reading will fall without the scan zone, meaning that homeostasis was not restored. If the aggregate reading falls without the scan zone, each of the remedies in the list are discarded by the computer as ineffective—step 368. At this point, the computer is directed to test the other segment of the split list—step 372 To test the other segment, the computer is directed to proceed again to step 346, where the remaining segment is split again according to pre-determined criteria. This process is repeated until a most appropriate product/remedy is discovered. This product/remedy is then stored in the holding tank database.

Following step 358, if the initial aggregate reading falls within the scan zone, thus signaling a restoration of homeostasis, the computer is directed to discard the other segment—step 362. The computer then is directed to determine if there are two or more products/remedies remaining in the segment, step 366. If so, this becomes the new list, step 376 and the computer is directed to proceed to step 346, where this new list is then split and the process repeats as described above. If there are not two or more products/remedies remaining, the computer has narrowed and singled out the most effective and appropriate product/remedy for restoring homeostasis. This product/remedy filter is then stored by the computer in the holding tank database and associated with an electromagnetic signature—step 380. At this point, the Remedy Scan Function is complete and deactivated. It should be noted that products/remedies that have already been placed in the holding tank are never included in any other remedy scan function for that particular client during the same session.

Once the most effective or appropriate product/remedy filter has been identified as being the most beneficial to the patient, the product/remedy treatment can be administered to the patient in one of several ways: (1) an existing homeopathic of that product/remedy filter (homeopathic remedies are frequency specific) can be administered to the patient through the meridian linking device; (2) a homeopathic remedy comprising the electromagnetic signature of the product/remedy can be prepared by the Meridian linking system and given to the patient with specific prescription requirements, including dosage and duration; and (3) the individual can be placed within the transmitters range and receive the product/remedy filter directly.

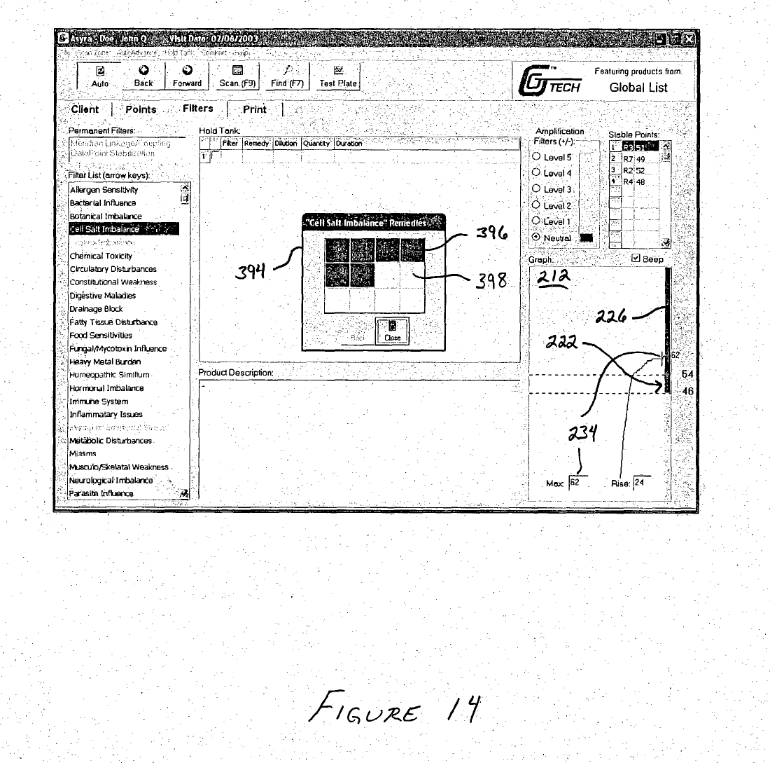

FIG. 14 illustrates a screen-shot of an exemplary operating environment during a Remedy Scan Function. Specifically, FIG. 14 illustrates the auto advance mode of the Remedy Scan Function, wherein product/remedy display field 394 shows the current product/remedy filter as it is being tested to determine its effectiveness against the broadcast customized filter (shown as the cell salt imbalance filter) that created an imbalance in the patient. The graphical data representing the tested test point value 234 of customized filter is shown in point status indicator field 212 as the filter is shown falling without scan zone 222, and particularly above the upper parameter and within upper imbalance field 226. Each time the initial step of the Remedy Scan Function is initiated, each product/remedy selected for the testing process is randomly assigned a space within the product/remedy display field. The product/remedy remains assigned to that space until the testing process is complete. When, or if, a new remedy scan is desired, each product/remedy is again assigned a random space. This random assigning helps to ensure testing objectivity as the user is unable to guess or memorize which space represents a particular product/remedy. Product/remedy display field displays active product/remedy signatures 396 that are being evaluated by the current step of testing and inactive product/remedy signatures 398 that are not being evaluated by the current step. Product/remedy display field may also comprise other unused areas that indicate that no product/remedy has been assigned to that position.

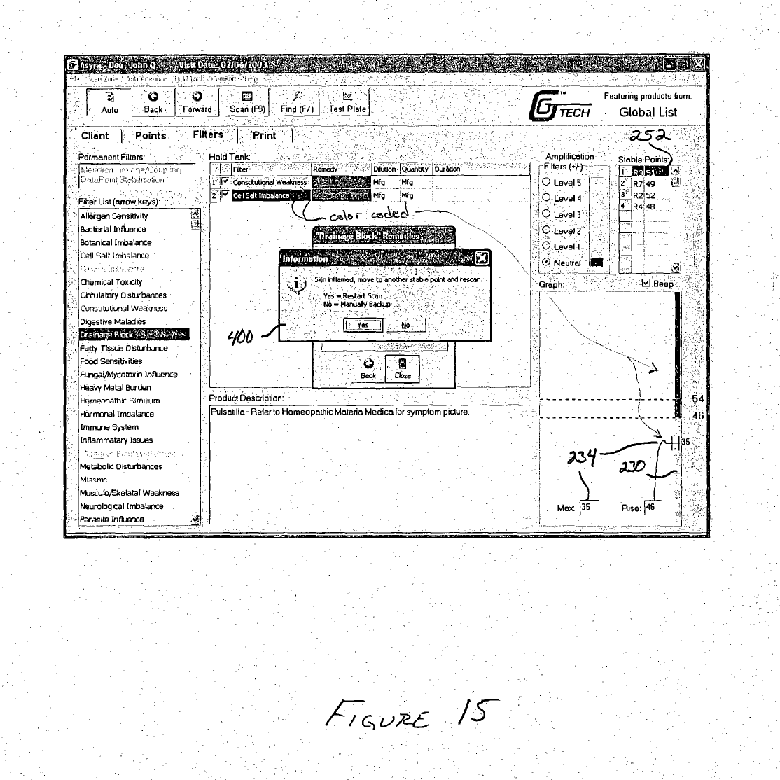

Normally the Remedy Scan Function will cause the computer to advance through the list of products/remedies without error. However, occasionally the skin surrounding the reference point being used for testing will become inflamed, thus skewing or distorting the results of the test. If this happens, the meridian linking system is designed to alert the user so that the error can be corrected, such as by moving to another stable reference point. In fact, it is preferable that a number of stable reference points be utilized throughout the diagnosing and treatment session. The error may appear for other reasons, such as a result of user error, wherein an improper reading of the stable reference point is taken. Either way, the user can just rescan to correct the error. FIG. 15 illustrates a screen shot of an exemplary warning or notification 400 that may appear during any process or function being performed in treating the patient. As a side note, FIG. 15 also illustrates how each customized filter stored in the holding tank may be coded (e.g., color coded) so that the user may ascertain at a glance the general type of imbalance of that particular customized filter.

### Amplification Function

One of the unique features of the present invention is its computer software Amplification Function. The Amplification Function causes the computer to increase the intensity/amplitude of each customized filter, thus increasing the stress put on the meridian network that is linked to the several internal organs, tissues, and systems in the patient. Increasing the intensity of the filter allows the body to reveal any hidden weaknesses that can become manifested with increased stress of the patient, which stress may be caused by work, the environment, poor diet; inherited weaknesses, etc. stated differently, increasing the intensity of the filter functions to induce or generate an increased stress within the body of the patient, thus allowing the user to diagnose and reveal latent maladies or even subtly manifested maladies that would otherwise be hidden and go unnoticed. It should be noted that not all amplification levels will cause an imbalance. Applying amplification levels to the customized filters however, provides the user with a significant tool for revealing and diagnosing latent maladies, such as dormant pathogens, deep emotional issues, hidden toxins, and allergies, etc. Prior art EAV, EDS, or GSR devices are incapable of such functions.

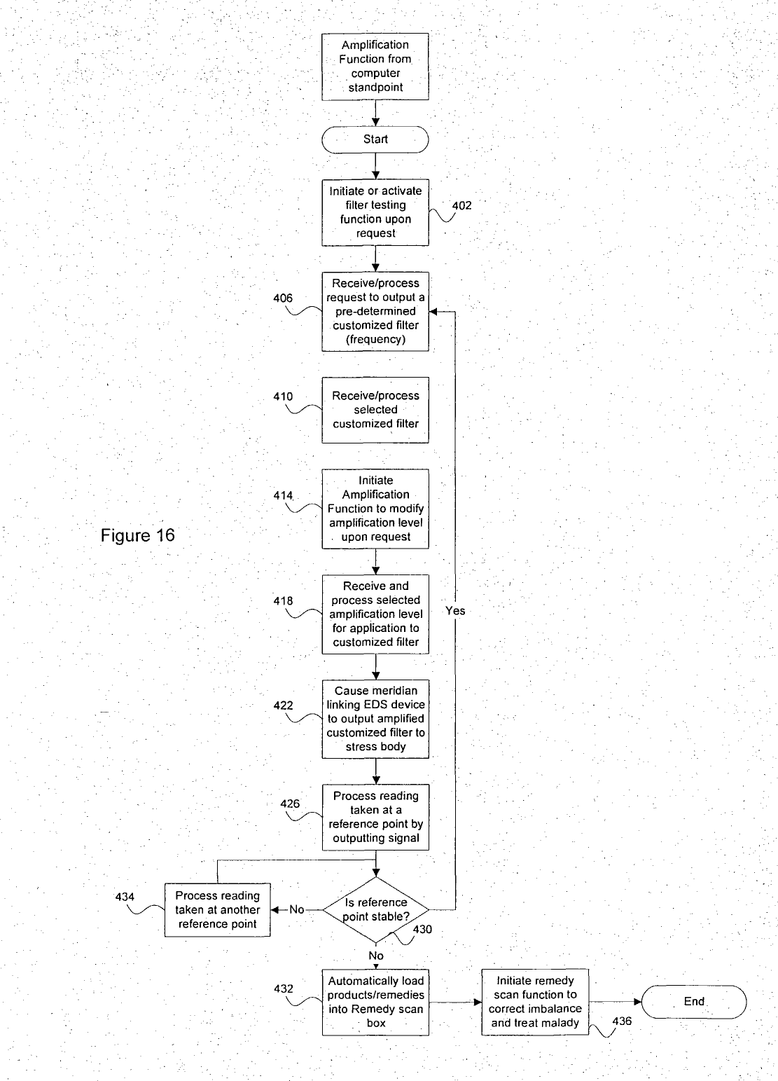

With reference to FIG. 16, once the computer system activates or initiates the Filter Testing Function as requested or directed by the user—step 402, the computer receives a request from the user for the computer to output or broadcast a pre-determined customized filter—step 406. As mentioned, filters are designed to stress the body with certain conditions. If the body can maintain homeostasis, the reading will stay within the scan zone. However, if any internal bodily physiology responds adversely, thus indicating an existing or underlying or latent malady, an imbalanced reading (either above or below the scan zone), will result.

Once the desired filter is selected by the user, the computer processes this request, step 410, which includes retrieving the filter from the filter database, and causes the meridian linking device, via the Filter Testing Function, to output or broadcast the selected customized filter for the purpose of stressing the body—step 262. Prior to broadcasting the signal or filter, the user can modify the intensity/amplitude of the filter by initiating the Amplification Function, step 414, wherein the computer is directed to receive and process a request from the user to modify the amplification level, as requested by the user, for application to the selected customized filter—step 418. Once processed, the Amplification Function directs the computer to instruct the meridian linking device to output the filter with the new modified amplification level—step 422.

Step 422, and particularly the application of the amplification level to the customized filter may be accomplished in various ways. For example, each amplification level may be selectively applied to the customized filter via manual selection by the user. Once a level is selected, a reading is taken at the stable reference point to determine if an imbalance is created. Each amplification level may therefore be tested through. Another example is using a more automated approach. Amplification Function may also direct a computer to set the amplification level for all customized filters that are selected for testing. In this mode, each customized filter broadcast by the meridian linking device will comprise a modified amplification level based on that selected.

As the filter is being broadcast, the user then takes a reading at one of the stable reference points via the permanent point stabilizing frequency, wherein the reading is sent back to the computer for processing—step 426. If any of the organ/tissue sets are not affected, the reference point will measure within the scan zone, which indicates that the particular customized filter chosen was not associated with any malady in the patient's body. Thus, the user selects another customized filter and the computer processes this request, step 410, to cause the meridian linking system to output the newly selected filter. This process can be repeated as many times as there are customized filters.

However, if any of the organ/tissue sets are affected it will change the overall resistance of the body network and the meridian linking system will detect and measure the change at the measured stable reference point(s)—step 430. To make sure the resistance of the body did actually change and not the stability of the reference point, a second measurement can be taken at another reference point, wherein the computer processes this reading and returns the measured resistance value—step 434. A reading outside of the scan zone indicates an imbalance in the meridian network, and thus the organ/tissue set, and that the body's energetic balance can be restored by the use of the specific filter that was used to cause the network to change. Once an imbalance is discovered, the computer automatically loads the products/remedies in the scan box—step 432. At this time, the computer also initiates or activates, either automatically or at the request of the user, the Remedy Scan Function of the meridian linking system—step 436. In this embodiment, storing the customized filter in the holding tank also comprises associating and storing the amplification level therewith.

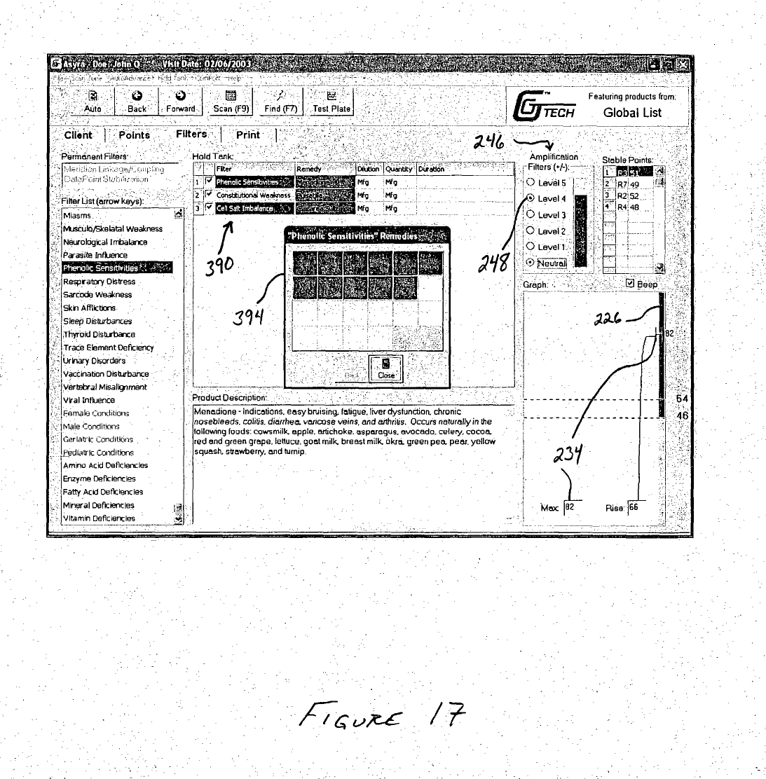

FIG. 17 illustrates a screen-shot of an exemplary operating environment, wherein amplification field 246 shows that the “phenolic sensitivities” filter has been modified to increase its intensity/amplitude to level four. Each amplification level is predetermined and stored by the computer, for later selection by the user.

### Prescription Attributes and Prescription Constraint Function

The present invention meridian linking system further features several prescription attributes associated with or assigned to the product/remedy filter chosen for restoring balance in and treatment of the patient via a Prescription Constraint Function. Prescription attributes facilitate safe, effective treatment or administration constraints that allow the product/remedy to be administered in a safe, effective manner. In a preferred embodiment, the prescription attributes are comprised of dilution constraints, quantity or dosage constraints, and duration of administration constraints, each comprising filters or frequencies that may be attached to the various products/remedies stored in the holding tank as dictated by the user. For each product/remedy selected, the user subsequently selects the specific constraints to be associated with that particular product/remedy via a Prescription Constraint Function comprising a Dilution Function, a computerized Dosage Function, and a computerized Duration Function, respectively, wherein each respective Function causes the computer to assign one or more prescription constraints to a product/remedy. These constraints can be assigned to a single remedy, multiple remedies, or all remedies at a time. It should be noted that any product/remedy selected in the holding tank will cause that product/remedy, or rather its filter or frequency, to be continually broadcast until unselected; Once selected, the dilution, dosage, and duration constraints can be tested against these broadcasted signals. The output of selected products/remedies can be used to develop customized homeopathic solutions.

### Dilution Function

The present invention further features a computer software Dilution Function that tests for the dilution of the product to be administered, wherein the dilution electromagnetic signal is located on an energy scale. Once one or more remedies have been selected, the Dilution Function is activated by the user. The Dilution Function causes the computer in the meridian linking system to dictate the dilution of the treatment administered to the patient.

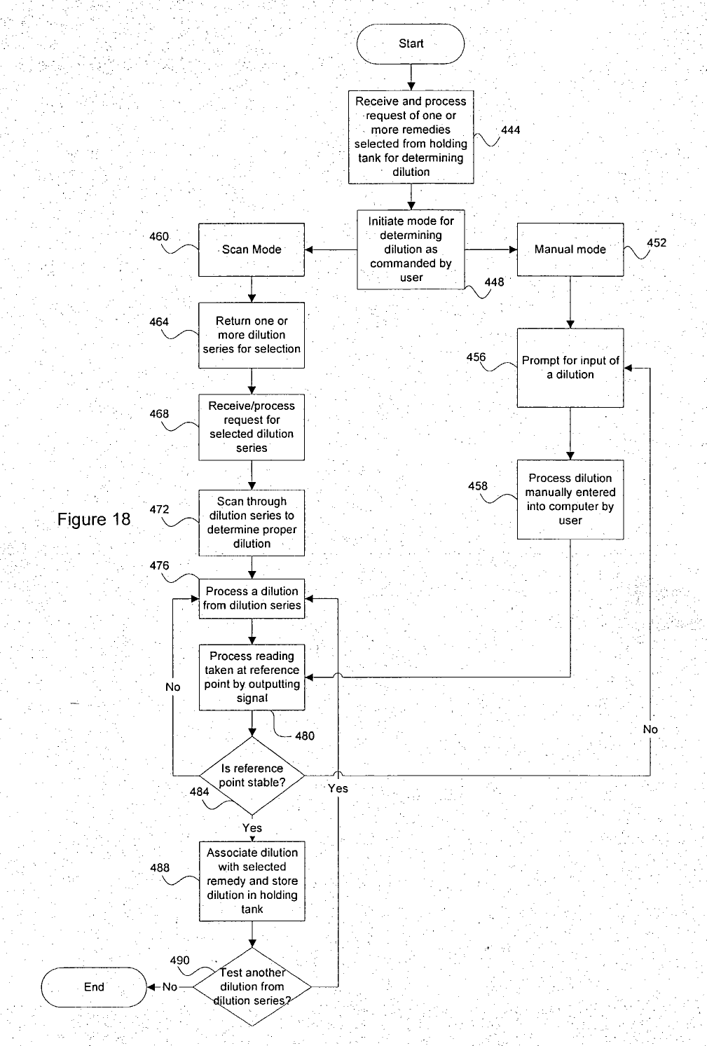

With reference to FIG. 18, step 444 comprises the computer receiving and processing a request for one or more products/remedies selected from the holding tank to receive a dilution constraint. A specific dilution may be assigned using a manual or automatic process, wherein each one is activated by the user, thus causing the computer to initiate that respective mode—step 448. In the manual mode, shown as box 452, the Dilution Function causes the computer to prompt the user to enter a specific dilution for the selected remedy—step 456. The user then enters a specific dilution to be applied to the selected product/remedy and the computer processes this dilution constraint, which comprises temporarily assigning it to the product/remedy—step 458. A test of the stable reference point is then taken and received and processed by the computer, step 480, to determine if the assigned dilution returns a stable point reading, or in other words, restores homeostasis—step 484. If the reading is stable, the Dilution Function causes the computer to assign or associate the given dilution with the selected remedy as part of the product/remedy electromagnetic signature and to store the dilution in the holding tank—step 488. From here, the user can select another product/remedy and repeat the above process. If, however, the reading is not stable, the Dilution Function causes the computer to discard that dilution constraint and to prompt the user to enter another dilution and to repeat the process beginning with step 456.

In scan mode, shown as box 460, the Dilution Function causes the computer to return one or more dilution series for selection by the user—step 464. A dilution series is defined herein as a plurality of pre-determined dilution constraints that may be assigned to the selected products/remedies. The user selects a dilution series and the computer receives and processes this request—step 468. The Dilution Function then causes the computer to scan through the dilution series to determine the proper dilution to be assigned or associated with the selected products/remedies—step 472. This is accomplished by the computer processing a given dilution as selected by the user—step 476. The frequency or filter of the selected dilution is then broadcast and a reading taken at a stable reference point to determine if the dilution is within or without the stable range. The Dilution Function causes the computer to receive and process this reading, step 480, and to determine whether the reference point is stable—step 484. If the reference point is stable, the Dilution Function causes the computer to associate or assign the dilution constraint to the product/remedy and store the dilution constraint in the holding tank as part of the electromagnetic signature of the product/remedy—step 488. Other dilution series may also be tested using the same procedure—step 490. If the point is not stable, the Dilution Function causes the computer to process another dilution series,

### Dosage/Quantity Function

The present invention further features a computer software Dosage/Quantity Function (hereinafter Dosage Function). Once one or more remedies have been selected, and once the dilution constraints have been assigned, the Dosage Function is activated by the user. Appropriate dosages are tested by sending out the electromagnetic signal, one to numerous times, within a specific time reference.

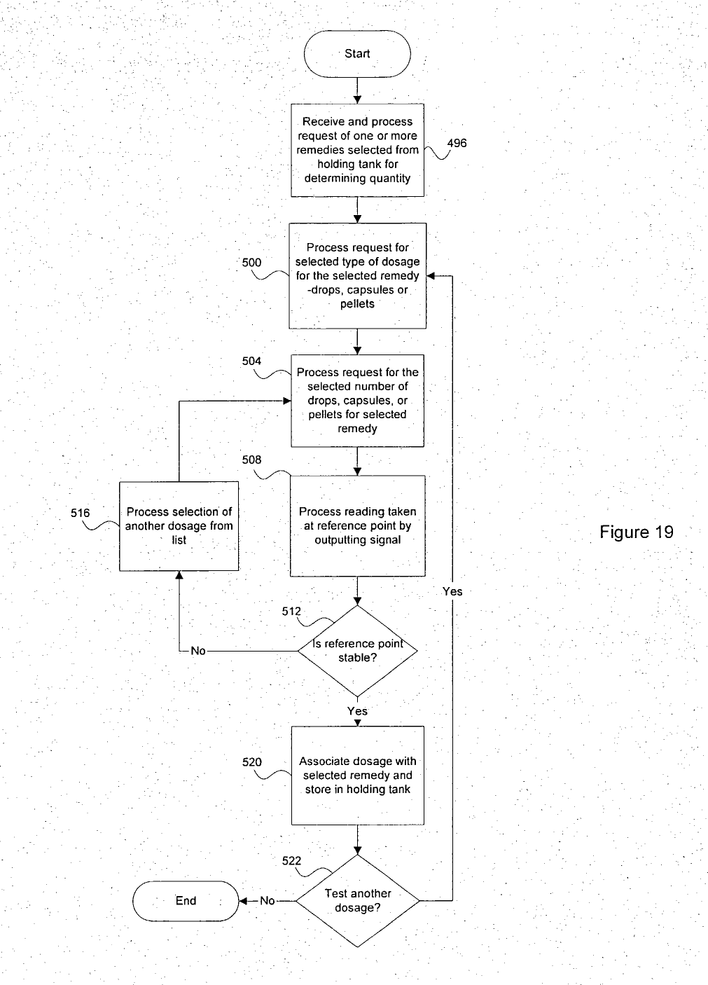

With reference to FIG. 19, the Dosage Function causes the computer in the meridian linking system to receive and process a request from the user of one or more products/remedies stored in the holding tank for determining the appropriate dosage to be administered to the patient—step 496. Once one or more remedies/products are selected, the Dosage Function causes the computer to process the selected type of dosage to be attributed to the products/remedies—step 500. Types of dosages include various administration mediums, such as pills, lozenges, capsules, pellets, and other similar or commonly known mediums. Once the type is selected by the user and processed by the computer, the user then selects the quantity to be administered. The computer processes the selection by the user, step 504, wherein the selection may be entered manually or selected from a list of pre-determined dosage amounts. To evaluate the effect of the dosage, the type and quantity are selected as indicated above. A reading is then taken at the stable reference point by outputting or broadcasting a signal comprising the frequency of the dosage constraints, wherein the reading is received and processed by the computer, step 512, to determine whether the reading is stable or not—step 512. If the reading is stable, the dosage constraints are assigned to the selected products/remedies and stored in the holding tank as part of the products/remedies electromagnetic signature—step 520. From this point, other dosages or dosage constraints may also be tested using the same process to acquire different dosages for as many products/remedies as desired. If the reading at step 512 is not stable, the dosage is discarded and another chosen for testing—step 516. The process then repeats to test the newly selected dosage. It is possible that several different dosages will achieve balance. When this occurs, the user simply may choose which dosage to assign to the selected products/remedies.

The computer processes instructions from the user to test other dosages or not step 522. If so, the process repeats from step 500 until no other dosages are desired for testing. If the user does not wish to test another dosage, the Dosage Function is deactivated.

### Duration Function

The present invention further features a computer software Duration Function. Once one or more remedies have been selected, and once the dilution and dosage constraints have been assigned, the Duration Function is activated by the user.

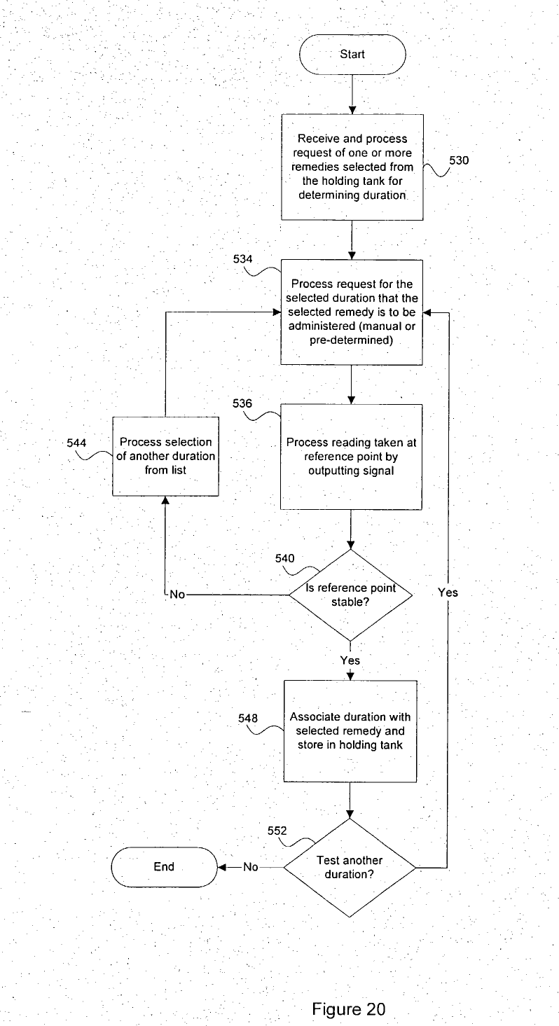

With reference to FIG. 20, the Duration Function causes the computer in the meridian linking system to receive and process the selection of one or more products/remedies as selected from the holding tank by the user—step 530. Once one or more products/remedies are selected, the Duration Function causes the computer to receive and process a request for the duration that the selected products/remedies are to be administered for treatment of the patient—step 534. The duration of administration may be entered into the system manually, or the system may comprise a plurality of pre-determined duration constraints selectable by the user. Once a duration constraint is selected or otherwise entered into the system, the computer receives and processes a reading taken at a stable reference point by outputting a signal comprising the frequency corresponding to the duration constraint—step 536. As part of the processing step, the computer then determines whether the reference point is stable—step 540. If the reference point is stable, the duration is appropriate and will be effective at restoring homeostasis in the patient and the duration is associated with the products/remedies and stored in the holding tank as part of the electromagnetic signature of the products/remedies—step 548. If the reference point is not stable, another duration is selected and processed by the computer via the Duration Function, step 544, and the process repeated. The above process may be repeated as often as necessary for additional duration constraints—step 552.

To test for the most effective or appropriate duration, several different testing standards or benchmarks may be used. In one exemplary embodiment, biological age filters are utilized. These filters allow the user to determine the appropriate length of time each product/remedy should be administered based upon the age of the patient. Other standards might include or be based upon the patient's fitness level, gender, past reactions to certain treatments, size and weight, etc.

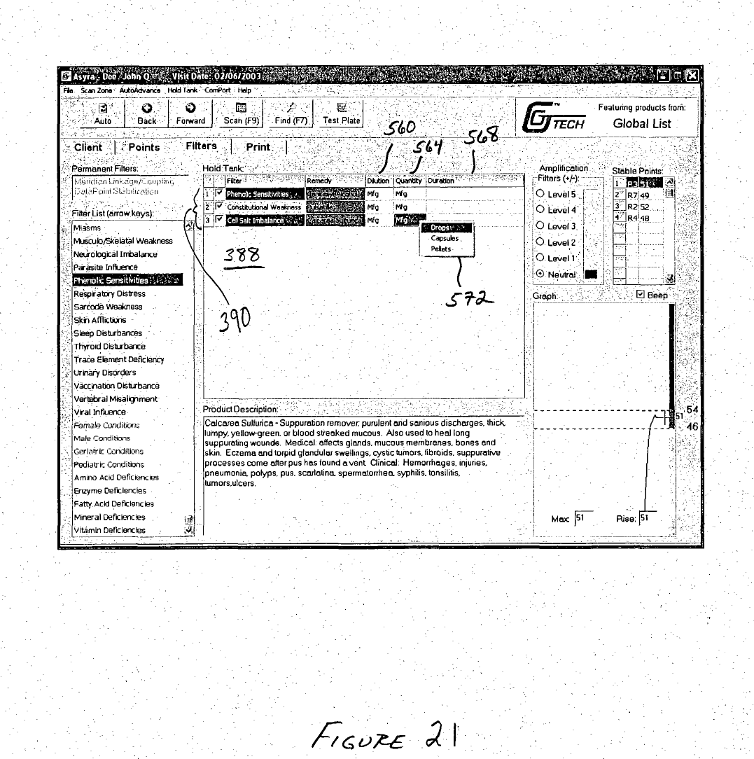

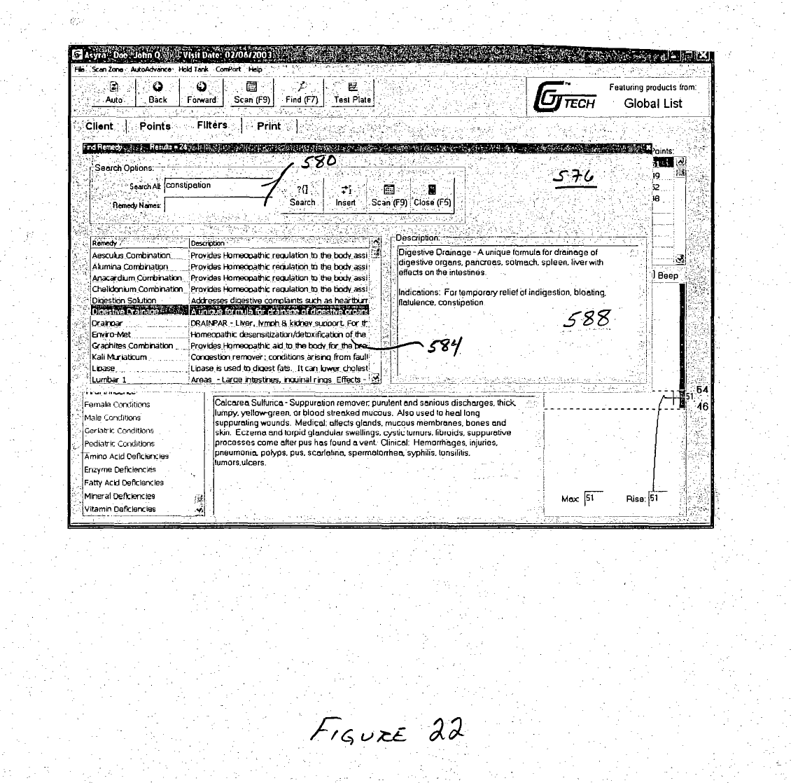

FIG. 21 illustrates a user interface screen-shot featuring an activation sequence for the Dosage Function of the computerized meridian linking system, according to one exemplary embodiment of the present invention. As shown, holding tank 388 comprises several products/remedies 390 that are selected to receive dilution 560, dosage 564, and duration 568 constraints. Specifically, FIG. 21 illustrates duration constraints 564 being selected from a pre-determined dosage list 572 comprising drops, capsules, or pellets. Although not shown, similar activation sequences are contemplated for each of the other constraints discussed herein.

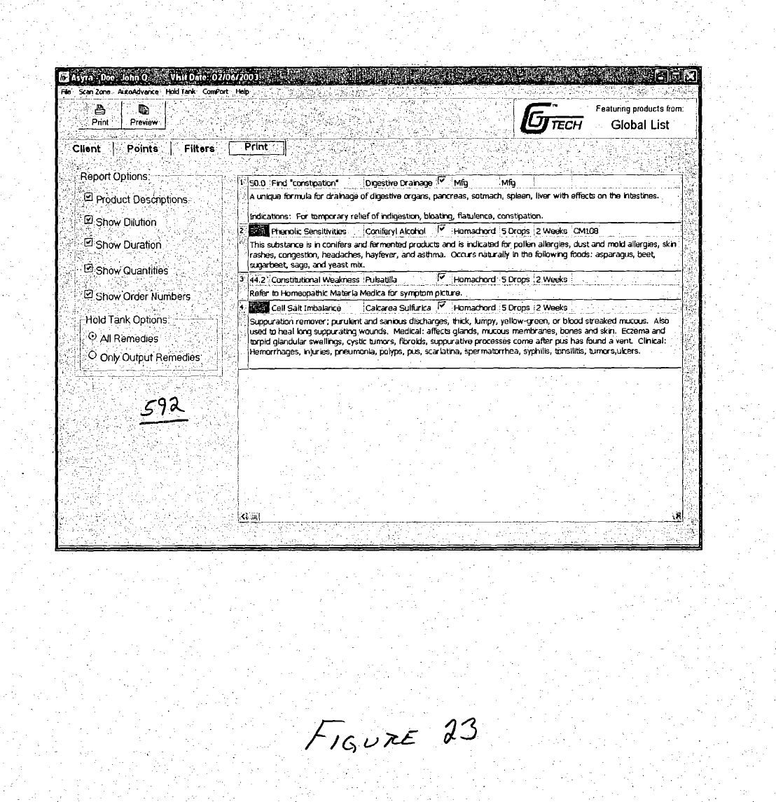

FIG. 22 illustrates a user interface screen-shot of the search environment 576 featuring activation of the manual remedy search function of the computerized meridian linking electrodermal screening diagnostic system, according to one exemplary embodiment of the present invention. Since the meridian linking system contains a database of all of the names and product descriptions, shown in product/remedy field 584 and product description field 588, respectively, of the products/remedies, the search function allows the user to search the database of product/remedies that contain the words or symptom the user wishes to locate as entered into query 580. Advantageously, the user is able to search both the product descriptions and the actual product/remedy names. The search function creates a list, shown in product/remedy field 584 of the products/remedies that match the criteria entered by the user in the search query. The search function utilizes standard searching algorithms as commonly known in the art. Once the results from the search are returned, the user can select the product/remedy and test the patient's response to the product/remedy. In addition, the user can activate the scan feature to automatically scan for the most effective or appropriate remedy as describe above.

With reference to FIG. 23, shown is a reports and printing environment 592, wherein the products/remedies stored in the holding tank can be printed, along with their product descriptions, for later review. Various reports can also be generated and printed, each based on user selected criteria.

### Test Plate Function

The present invention further features a computer software Test Plate Function for testing products for both effectiveness and tolerance with respect to a particular patient. The Test Plate Function of the meridian linking system provides the ability to test the effectiveness and appropriateness of products/remedies that are not initially entered and stored within the product/remedy database against those customized filters creating an imbalance within the meridian network. First, the computer receives and process a request to activate, and then activates, the Test Plate Function—step 594. The Test Plate Function subsequently causes a computer to receive and process the frequency generated by the product placed on the test plate—step 596. At this point, the user selects one or more customized filters from the filters list that have been tested to cause an imbalance in the meridian network to test against the product on the test plate. The Test Plate Function then causes the computer to instruct the meridian linking device to output or broadcast the selected customized filter(s), step 598, thus inducing an imbalance in the meridian network of the patient. The computer then causes the meridian linking device to output the frequency of the product on the test plate so that it may be evaluated for effectiveness, or to see if it is beneficial to the patient—step 600. The product on the test plate is tested against the customized filter and is tested for both effectiveness and compatibility with the patient, each of these being tested for based on filters or frequencies broadcast corresponding to each, respectively. A reading at the stable reference point is taken and received and processed by the computer, step 602, wherein the computer determines whether the reading is within the stable point range or not—step 604. If the reading is stable, the product is deemed beneficial to the patient. At this time, the Dilution, Dosage, and/or Duration Function(s) may be activated to obtain prescription information as described above—step 606. With regards to the quantity of dosage of the product on the test plate, this can be determined without opening the product and manually placing samples on the test plate. The meridian linking system tests for quantity by sending out the electromagnetic signal, one to numerous times, within a specific time reference. No other prior art system is capable of doing this. The product filter and any prescription information are then caused to be stored in the system database—step 608. If the reading of the stable point at step 604 is not stable, the product is considered not beneficial to the client and discarded—step 610.

FIG. 25 illustrates a screen-shot of an exemplary test plate environment 640 comprising, among other things, an effectiveness/toleration filter activation status field 644. From this environment, the Test Plate Function is controlled, including the broadcasting of various customize filters and assignment of any prescription constraints.

### Imprinting Function

The present invention further features a computer software Imprinting Function. The Imprinting Function of the meridian linking system is an extension of the Frequency Testing Function and provides the ability to create a tangible homeopathic remedy of the product/remedy filter found to be most appropriate and effective for treatment of the diagnosed malady. Specifically, the Imprinting Function facilitates the transfer of an electromagnetic signature of a product/remedy from the meridian linking system's holding tank (database of stored products/remedies and their electromagnetic signatures) to a liquid medium via the test plate. The electromagnetic signatures are stored in the database of the meridian linking system as digital representations of the effect of a product/remedy on a biological entity.

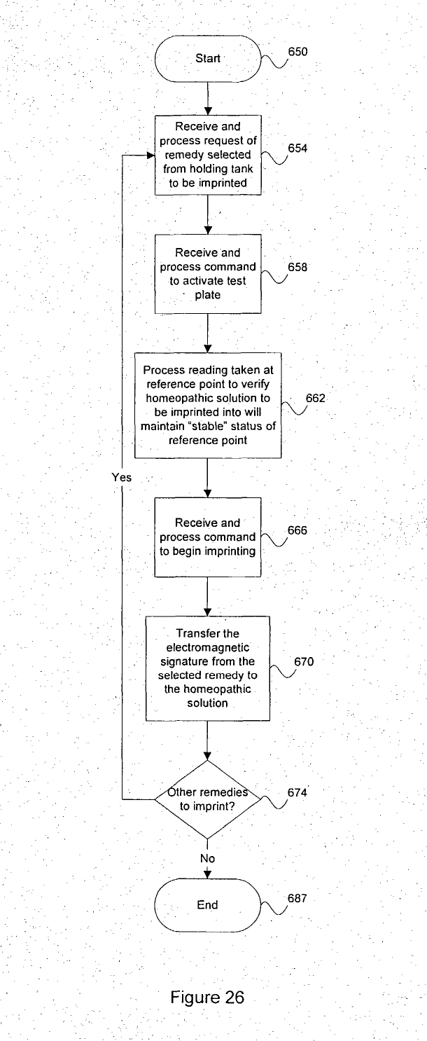

With reference to FIG. 26, the Imprinting Function causes the computer to receive and process the remedy, and preferably the most appropriate and/or effective remedy, selected from the holding tank by the user that is to be imprinted—step 654. A homeopathic solution or remedy is then placed on the test plate and the Test Plate Function activated and a reading at the stable reference point taken, wherein the computer determines whether the homeopathic remedy is capable of maintaining the stable status of the point—step 662. If so, the computer receives and processes a request by the user to begin imprinting, wherein the computer initiates the imprinting process, step 666, and transfers the electromagnetic signature form the selected remedy to the homeopathic solution. The computer then allows the user to select other or additional remedies for imprinting—step 674. If there are, the process repeats, beginning at step 654. If not, the Imprinting Function is deactivated. Using the product/remedy filter found to be most beneficial in restoring energetic balance to the individual, the meridian linking system intensifies the filter signal and sends it to an antenna or plate to be broadcast directly to the individual.

During the actual imprinting process, the meridian linking system intensifies the product/remedy filter and imprints it and its electromagnetic signature onto what is called a homeopathic blank, which is a solution comprised of alcohol and water. This process imprints or imparts the energetic frequency into the blank,; which can now be administered to the patient for restoring balance or homeostasis in the patient and for treating the diagnosed malady. It is preferred that the product/remedy filter and associated electromagnetic signature be transferred to an existing homeopathic remedy. This will not erase the existing homeopathic signatures, but will add the new signatures to the homeopathic solution. The signatures are stabilized/stored between the carbon bonds of the alcohol and water. Therefore, it is important that the homeopathic contain a minimum concentration of alcohol. In a preferred embodiment, alcohol is present in an amount between about 20 and 30 percent by weight. Indeed, the now energetic remedy may be administered to the patient to create or induce a balancing effect on the overall energetic status of the patient.

An imprinted remedy generated by the present invention meridian linking system contains energy signatures of higher or increased quality and magnitude than many similar prior art devices, thus resulting is decreased dosage, dilution, and/or duration of administration requirements.

### Methods of Operating the Meridian Linking System and Treating a Patient

The present invention further features several methods of operating the meridian linking system and for diagnosing and treating a patient. The methods are briefly discussed herein, and are more clearly understood when read in conjunction with the disclosure and corresponding Figures discussed above.

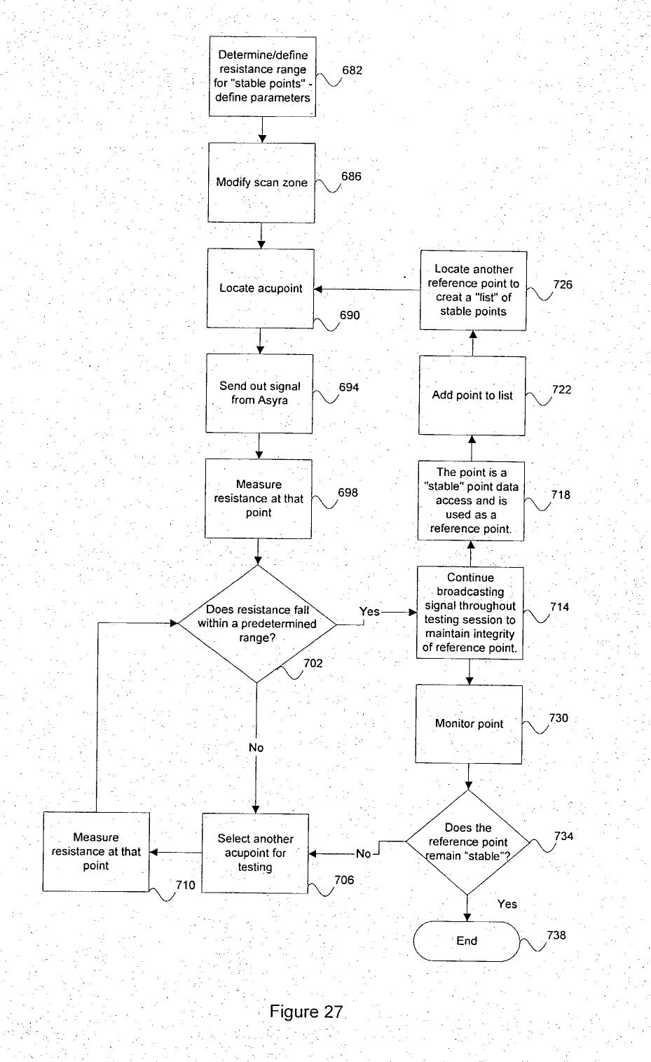

With reference to FIG. 27, the present invention features a method for linking the meridians in the body to establish an interconnected network, as well as a method for establishing a plurality of stable reference points linked to the interconnected meridian network. Specifically, the method comprises the steps of: step 682, determining or defining a resistance range or resistance parameters to identify a scan zone for “stable” points; step 686, modifying the scan zone if necessary; step 690, locating one or more acupoints or data access points; step 694, sending out a stabilizing signal from the meridian linking system; step 698, measuring the resistance at that point; step 702, determining whether the measured resistance falls within a predetermined range; step 706, selecting another data access point from the list of stable reference points because the measured resistance is without the range; step 710, measuring resistance at the newly selected data access point and repeating steps 702 and 706; step 714, continuing broadcasting the signal throughout the testing session to maintain the stable integrity of the reference point if the reading is within the stable points range; step 718, identifying the point as a “stable” data access point, wherein it may be used as a reference point; step 722, adding the reference point to a list of stable reference points; step 726, locating another reference point to create the “list” of stable points; step 730, monitoring the stable point throughout the testing and treatment session to maintain its status as a stable point; step 734, determining whether the reference point remains stable; and repeating steps 706, 710, and 702 if the reference point does not remain stable.

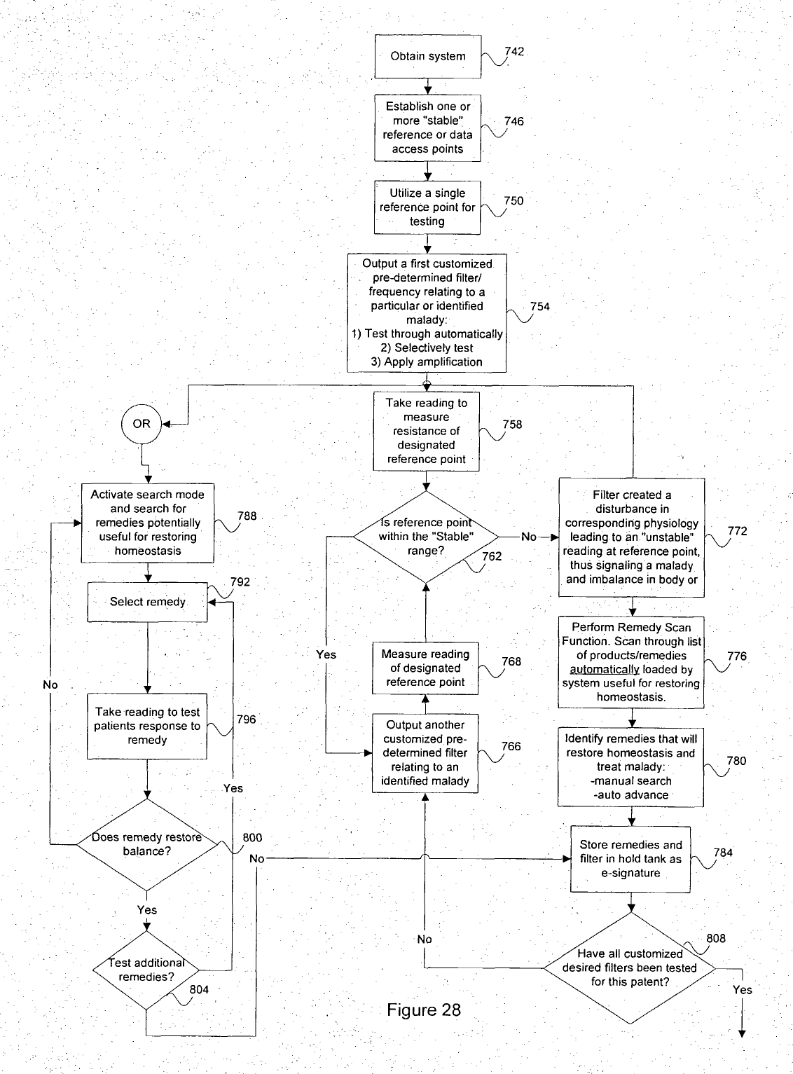

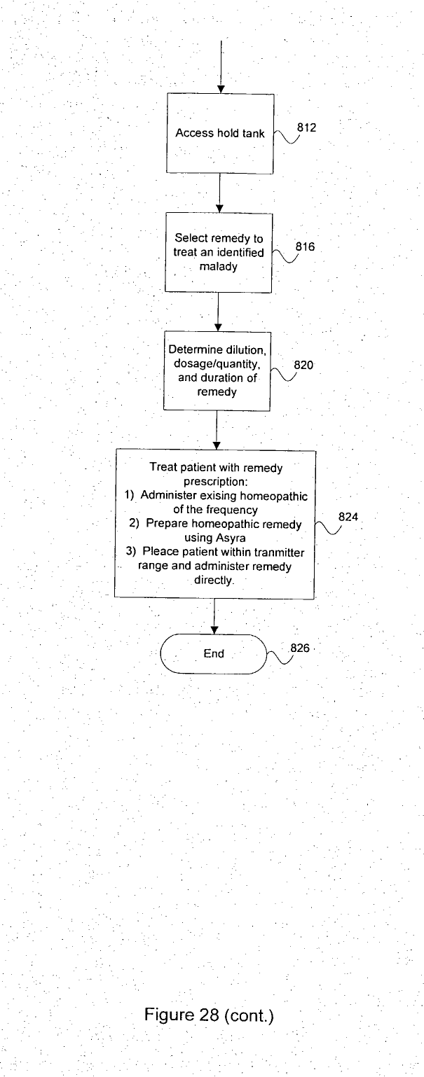

With reference to FIG. 28, the present invention also features a general method for diagnosing a malady and for treating the malady comprising the steps of: step 742, obtaining a meridian linking system, such as the one described herein; step 746, establishing one or more “stable” reference or data access points and compiling these in a stable points list; step 750, utilizing a single reference point for testing and treatment, wherein the single reference point is selected from the list of stable reference points and may comprise any one of these throughout the testing and treatment session; step 754, outputting one or more customized, pre-determined filters/frequencies relating to a particular or identified malady, wherein the customized filters may be test through automatically, selectively by the user, or an either of these with an applied amplification; step 758, taking a reading to measure the resistance of a designated reference point; step 762, determining whether the reference point is within the “stable” range; step 766, outputting another customized predetermined filter relating to an identified malady if reference point is stable; step 768, measuring the reading of the designated reference point and proceeding again to step 762; if the reference point is not stable, the filter created a disturbance in the corresponding physiology or meridian network, thus signaling a malady and imbalance in body shown as box 772. From here, the user can either advance to the following steps, namely step 776, which is performing or initiating the Remedy Scan Function to scan through list of products/remedies automatically loaded by system useful for restoring homeostasis; step 780, identifying remedies that will restore homeostasis and treat the discovered malady, using either a manual or automatic process; step 784 storing remedies and filter in hold tank along with associated electromagnetic signature. Alternatively, the user can advance from step 772 to the following steps, namely, step 788, activating a search mode and searching for products/remedies potentially useful for restoring homeostasis in the patient; step 792 selecting a remedy; step 796, taking a reading at a stable reference point to test patients response to the selected remedy; step 800, determining whether the remedy restores balance; step 804, determining whether additional remedies are to be tested; if no, repeating steps 788-800; if yes, step 808, determining whether all customized desired filters been tested for this patent; if no, repeating steps 792-804; if no, advancing to step 784; step 808, determining whether all customized desired filters been tested for this patent; if no, returning to step 766; if yes,-advancing to step 812, accessing the hold tank; step 816, selecting a remedy to treat the identified malady; step 820, determining dilution, dosage/quantity, and duration of the selected remedy to associate or assign prescription constraints to selected remedy; and step 824, treating patient with remedy according to associated prescription, wherein treatment may include administering existing homeopathic of the frequency, preparing a homeopathic remedy using the meridian linking system, or placing the patient within transmitter range and administering the remedy directly.

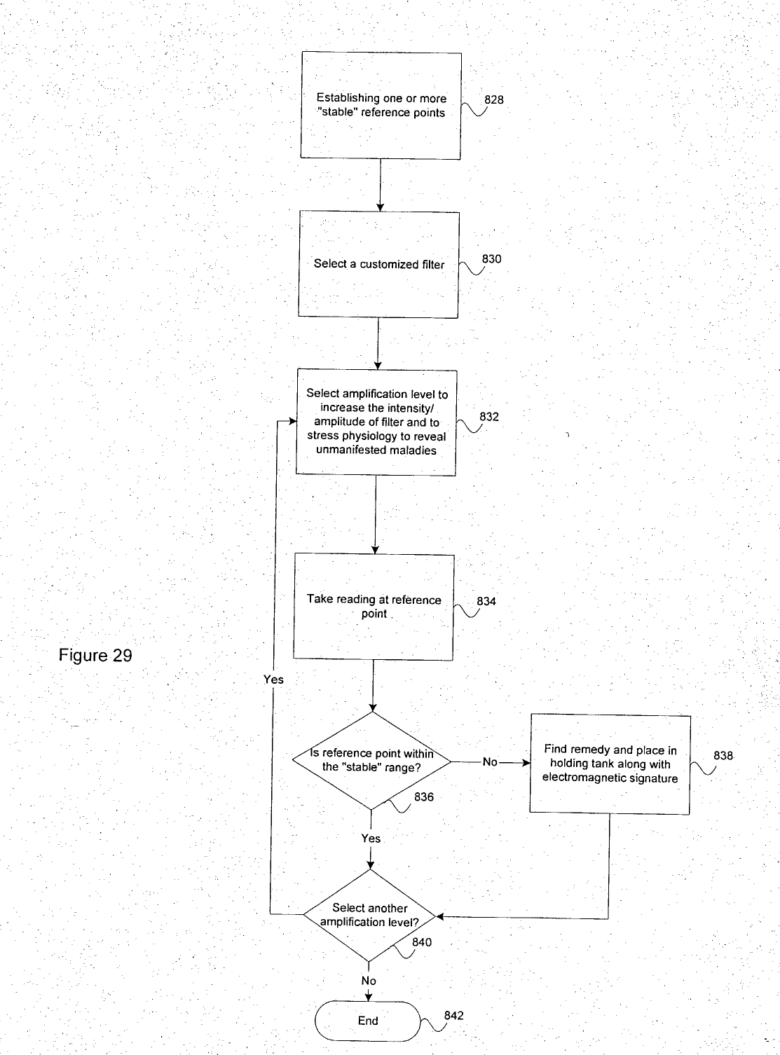

With reference to FIG. 29, the present invention further features a method for amplifying the intensity of the customized filter to reveal hidden or latent maladies, wherein the method comprises the steps of: step 828, establishing one or more “stable” reference points; step 830, selecting a customized filter to be broadcast; step 832, selecting an amplification level designed to increase the intensity/amplitude of the broadcast customized filter and to stress the physiology of the patient to reveal unmanifested, hidden, or latent maladies; step 834, taking a reading at a reference point; step 836, determining whether reading is within the “stable” range; if yes, advancing to step 840; if no, advancing to step 838, finding an appropriate and/or effective remedy and placing it within the holding tank, along with its associated electromagnetic signature; step 840, determining whether to select another amplification level; if yes, returning to step 832; and if no, advancing to step 842.

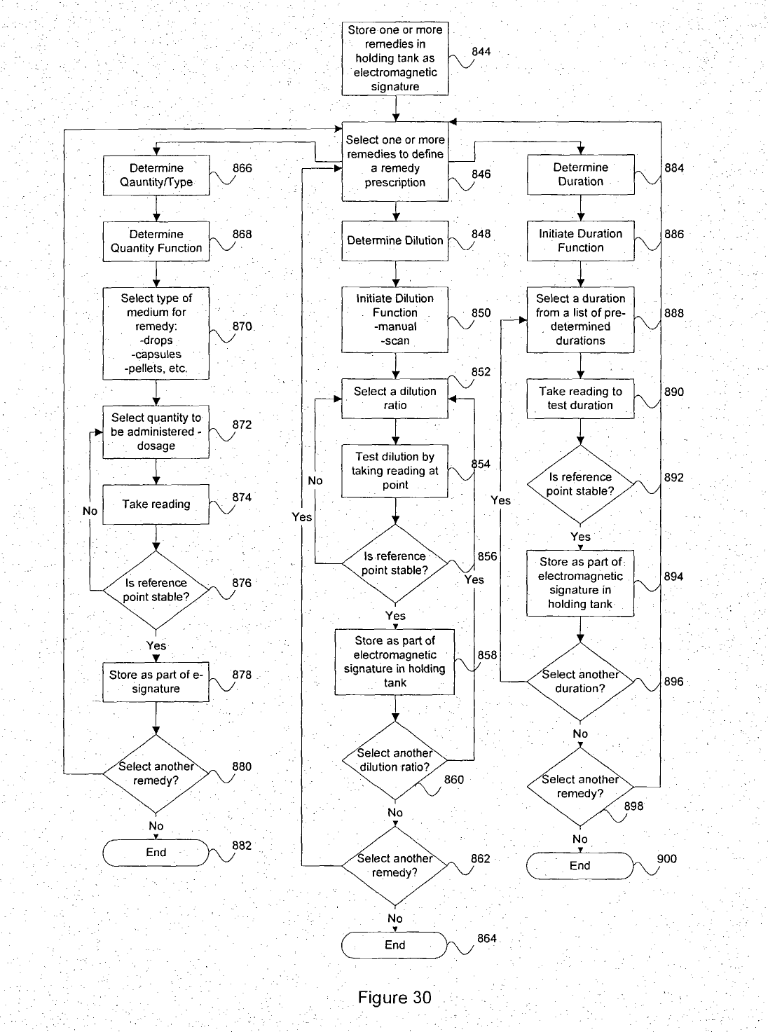

With reference to FIG. 30, the present invention further comprises associating prescription constraints to the one or more products/remedies selected for treatment of any discovered maladies, wherein the method comprises the steps of: step 844, storing one or more remedies in the holding tank, along with its associated electromagnetic signature; step 846, selecting one or more remedies to associate prescription constraints therewith; and associating a remedy prescription or associating prescription constraints, namely, dilution constraints 846, dosage constraints 866, and duration constraints 884.

The method for associating dilution prescription constraints comprises the following steps: step 850, initiating the Dilution Function; step 852, selecting a dilution ratio; step 854, testing dilution by taking reading a stable reference point; step 856, determining whether reading is within stable range; if no, returning to step 852, if yes, advancing to step 858, storing dilution as part of electromagnetic signature; step 860, determining whether to select another dilution ratio; if yes, returning to step 852; if no, advancing to step 862, determining whether to select another remedy; if yes, returning to step 846; and if no, advancing to step 864, the end.

The method for associating dosage prescription constraints comprises the following steps: step 868, initiating the Quantity/Dosage Function; step 870, selecting the type of medium for the selected remedy, namely drops, capsules, or pellets, etc.; step :872, selecting the dosage to be administered; step 874, taking a reading at the stable reference point; step 876, determining whether reading is within stable range; if no, returning to step 872; if yes, advancing to step 878, storing or associating dosage with remedy as part of electromagnetic signature; step 880, determining whether to select another remedy; if yes, returning to step 846; and if no, advancing to step 882, the end.

The method for associating duration prescription constraints comprises the following steps: step 886, initiating the Duration Function; step 888, selecting a duration from a list of pre-determined durations; step 890, taking a reading at a stable reference point to test duration; step 892, determining whether the reading is within the stable point range; if no, returning to step 888; if yes, advancing to step 894, storing duration as part of electromagnetic signature in holding tank; step 896, determining whether to select another duration; if yes, returning to step 888; if no, advancing to step 898, determining whether to select another remedy; if yes, returning to step 846; and if no, advancing to step 900, the end.

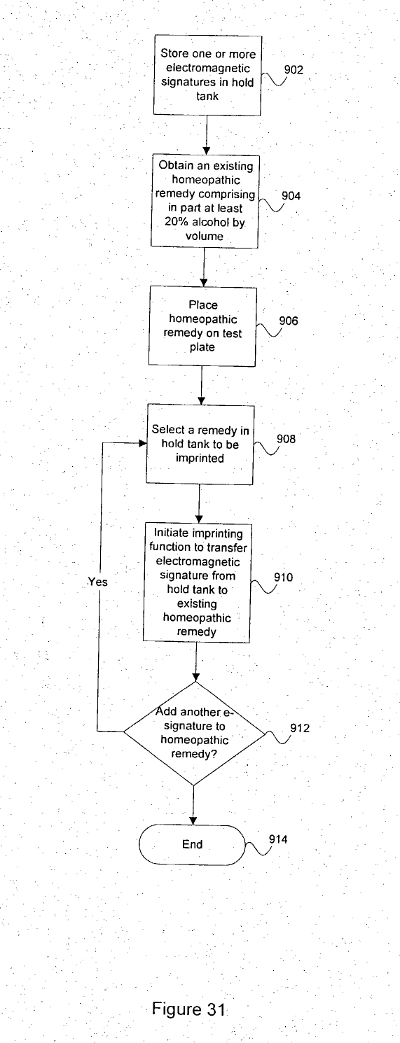

With reference to FIG. 31, the present invention further features a method for imprinting a remedy, with its associated electromagnetic signature, into an existing homeopathic remedy, wherein the method comprises the steps of: step 902, storing one or more remedies and corresponding electromagnetic signatures in the holding tank; step 904, obtaining an existing homeopathic remedy, preferably comprising in part an identified percentage of alcohol by volume; step 906, placing the homeopathic remedy on the test plate; step 908, selecting a remedy in the holding tank to be imprinted; step 910, initiating the Imprinting Function that transfers the frequency and electromagnetic signature of the remedy from holding tank to the existing homeopathic remedy; step 912, determining whether to add another electromagnetic signature to the homeopathic remedy; if yes, returning to step 908; and if no, advancing to step 914, the end.

The present invention may be embodied in other specific forms without departing from its spirit or essential characteristics. In addition, the described embodiments are to be considered in all respects only as illustrative and not restrictive. As such, the scope of the invention is indicated by the appended claims, rather than by the foregoing description. All changes which come within the meaning and range of equivalency of the claims are to be embraced within their scope.

What is claimed and desired to be secured by Letters Patent is:

1. A computerized meridian linking diagnostic and treatment system for diagnosing and treating existing and latent maladies within a patient, said system comprising:
(a) a computer and computer system capable of storing information thereon and executing one or more computer software functions that control said system, said computer software functions comprising:
a meridian linking function that links all the meridians in a patient to form an interconnected meridian network;
a data access point stabilizing function that establishes and stabilizes one or more data access points as stable reference points having resistance measurements within a predetermined stable range;
a filter testing function that utilizes customized filters to stress said meridian network to reveal manifested and latent maladies in said patient and to automatically load pre-determined products/remedies identified as effective against said maladies revealed by said customized filters;
an amplification function for varying the amplitude and intensity of said customized filter to reveal both manifested and latent maladies;
a remedy finding function that identifies and isolates a single, most effective and appropriate product/remedy from said loaded products/remedies;
a prescription function that assigns prescription constraints to said single, most effective and appropriate remedy; and
(b) a meridian linking diagnostic and treatment device controlled by the computer that outputs one or more filters to a patient through a meridian network, as directed by said computer software functions, using a single stable reference point.

2. A computerized meridian linking diagnostic and treatment system for diagnosing and treating existing and latent maladies within a patient, said system comprising:
(a) a computer and computer system capable of storing data and executing one or more computer software functions thereon;
(b) a meridian linking function that controls said computer to cause a meridian linking diagnostic and treatment device to output a filter that links all of the meridians within the body of a patient to create an interconnected meridian network linking all of the internal systems and organs within said patient to one or more data access points;
(c) a data access point stabilizing function that controls said computer to cause said diagnostic and treatment device to output a filter that detects and identifies whether said one or more data access points are stable to function as testing reference points, which, from any single said reference point, all of said internal systems and organs within said patient can be tested as they are linked via said meridian network, said data access stabilizing function also stabilizing said reference points within a predetermined stable range throughout a testing session; and
(d) a filter testing function for testing both existing and latent maladies in said patient, said filter testing function controls said computer to cause said diagnostic and treatment device to selectively output a customized malady-specific filter to said patient at an identified level to determine if said filter induces an imbalance in said meridian network, measured at said reference point, thus indicating a corresponding malady within said patient, said filter testing function further controls said computer to automatically load one or more products/remedies from a products/remedies database into a products/remedies list if said customized filter induces an imbalance within said meridian network, wherein said loaded products/remedies are pre-determined to correspond to said customized filter and one or more maladies;
(e) a remedy finding function that controls said computer to initiate an elimination process to determine which of said loaded products/remedies are the most effective and the most appropriate for treatment of said patient, wherein the remedy scan function focuses on narrowing a large list of potential treatment remedies down to the single, most effective and appropriate remedy; and
(f) a prescription function that associates prescription constraints with said single, most effective and appropriate remedy for proper treatment of and administration of said remedy to said patient.

3. The computerized meridian linking diagnostic and treatment system of claim 2, further comprising an amplification function that controls said computer to modify the amplitude and intensity of said customized filter to vary the stress induced within said meridian network by said customized filter, said amplification function facilitating diagnosis of both manifested maladies needing corrective treatment and latent maladies eligible for preventative treatment.

4. The computerized meridian linking diagnostic and treatment system of claim 2, further comprising a test plate function that controls said computer and said computerized diagnostic and treatment device to test said patient's effectiveness and tolerance of said products/remedies that are not initially entered and stored within said products/remedies database against said customized filters that create an imbalance within said meridian network as substantiated by a stable point test of said one or more reference points.

5. The computerized meridian linking diagnostic and treatment system of claim 2, further comprising an imprinting function that controls said computer and said computerized meridian linking diagnostic and treatment device to create a tangible customized homeopathic remedy of said single, most effective and appropriate product/remedy by facilitating the transfer of said filter and any associated electromagnetic signatures to an existing homeopathic remedy via a test plate, and as substantiated by a stable point test of said one or more reference points.

6. The computerized meridian linking diagnostic and treatment system of claim 2, wherein said prescription function comprises a dilution function that controls said computer to associate and assign dilution constraints to one or more said products/remedies as substantiated by a stable point test of said one or more reference points.

7. The computerized meridian linking diagnostic and treatment system of claim 2, wherein said prescription function comprises a dosage function that controls said computer to associate and assign quantity and dosage constraints to one or more said products/remedies as substantiated by a stable point test of said one or more reference points.

8. The computerized meridian linking diagnostic and treatment system of claim 2, wherein said prescription function comprises a duration function that controls said computer to associate and assign duration constraints to one or more said products/remedies as substantiated by a stable point test of said one or more reference points.

9. A computerized diagnostic and treatment system for diagnosing and treating existing and latent maladies within a patient, said system comprising:
(a) a computer and computer system capable of storing data and executing one or more computer software functions thereon;
(b) a meridian linking diagnostic device electrically coupled to said computer and comprising:
a primary base unit comprising at least one signal or filter generator therein that alternates broadcasting of a filter selected from a meridian linking filter, a point stabilizing filter, a malady-specific customized filter, a product/remedy filter, a prescription constraint filter, and a homeopathic remedy filter;
a stylus electrically coupled to said primary base unit that transfers said broadcast filter from said signal generator to said patient for reading and measuring the resistance at one or more data access points;
a control module located on said primary base unit for controlling several functions of said primary base unit;
(c) a meridian linking function that controls said computer to cause said meridian linking diagnostic device to permanently output said meridian linking filter, said meridian linking filter links all of the meridians within said patient, thus forming a single, interconnected network that links all of the internal systems and organs within said patient to said one or more data access points;
(d) a data access point stabilizing function that controls said computer to cause said meridian linking diagnostic device to permanently output said point stabilizing filter, said point stabilizing filter measures said one or more data access points to determine whether they fall within a pre-determined stable range, wherein said point stabilizing filter establishes and maintains those stable data access points as reference points during a testing session, which, from any single said reference point, all of said internal systems and organs within said patient can be tested;
(e) a filter testing function for testing both existing and latent maladies in said patient, said filter testing function controls said computer to cause said meridian linking diagnostic and treatment device to selectively output said malady-specific customized filter to said patient at an identified level to determine if said filter induces an imbalance in said meridian network, measured at said reference point, thus indicating a corresponding malady within said patient, said filter testing function further controls said computer to automatically load one or more products/remedies from a products/remedies database into a products/remedies list if said customized filter induces an imbalance within said meridian network, wherein said loaded products/remedies are pre-determined to correspond to said customized filter and one or more maladies;
(f) a remedy finding function that controls said computer to cause said meridian linking diagnostic and treatment device to output said product/remedy filters of said loaded products/remedies and to initiate an elimination process to determine which of said loaded products/remedies are the most effective and the most appropriate for treatment of said patient based on their resistance measurement, wherein the remedy finding function focuses on narrowing a large list of potential treatment remedies down to the single, most effective and appropriate remedy; and
(g) a prescription function that controls said computer to test for and associate stable prescription constraints with said loaded products/remedies, said prescription constraints providing proper treatment guidelines for administration of said remedy to said patient.

10. A computerized meridian linking diagnostic and treatment device comprising:
a primary control unit;
a computer interface contained within said primary control unit that interfaces with a computer and computer system to exchange information with said computer and to receive one or more commands from said computer to perform a specific task as directed by one or more computer software functions stored and executable on said computer;
a filter generator that continuously broadcasts two permanent filters, one of which establishes and maintains a plurality of stable reference points, and one of which links the meridians in said patient to form a meridian network linking all of the internal organs and systems to said one or more stable reference points, said filter generator also broadcasts one or more temporary filters;
a stylus electrically coupled to said primary control unit for facilitating the broadcasting of said filters to said patient via said stable reference points;
a control unit located on said primary control unit for controlling various functions of said device;

11. The device of claim 10, wherein said temporary filters are selected from the group consisting of a malady-specific customized filter, a product/remedy filter, a prescription constraint filter, a test plate filter, and a homeopathic remedy filter.

12. The device of claim 10, wherein said computer software functions are selected from the group consisting of a filter testing function, a remedy scan/finding function, an amplification function, a prescription constraint function, a test plate function; and an imprinting function.

13. A computer program product for linking the meridians within the body through a single data access point to form an interconnected meridian network, said computer program product comprising:
code that controls a computer to cause a meridian linking device to continuously output a permanent point stabilizing filter that establishes and maintains at least one data access point as a stable reference point having a resistance within a pre-defined stable range;
code that controls a computer to cause a meridian linking device to continuously output a permanent meridian linking filter via said stable reference point, said meridian linking filter forming a meridian network that links all of the internal systems and organs within a patient to a single stable reference point.

14. The computer program product of claim 13, further comprising code that controls a computer to cause a meridian linking device to output one or more temporary filters and to test and measure the resistance induced by said temporary filters at said single stable reference point.

15. The computer program product of claim 14, wherein said temporary filters are selected from the group consisting of a malady-specific customized filter, a product/remedy filter, a prescription constraint filter, a test plate filter, and a homeopathic remedy filter.

16. The computer program product of claim 13, further comprising code that controls a computer to signal an unstable reference point if said resistance of said reference point ever falls without said stable range, thus allowing utilization of another stable reference point.

17. A computer program product for diagnosing and treating one or more maladies in a patient, said computer program product comprising:
code that controls a computer to cause a meridian linking device to output a permanent point stabilizing filter that establishes and maintains at least one data access point as a stable reference point;
code that controls a computer to cause a meridian linking device to output a permanent meridian linking filter via said stable reference point, said meridian linking filter forming a meridian network that links all of the internal systems and organs within a patient to said stable reference point;
code that controls a computer to cause a meridian linking device to output remedy-specific customized filter;
code that causes a computer to automatically load and store within a product/remedy database one or more products/remedies corresponding to one or more of said customized filters that creates an imbalance in said meridian network;
code that causes a computer to identify from said loaded products/remedies a single, most effective and appropriate product/remedy capable of restoring homeostasis.

18. The computer program product of claim 17, further comprising code that controls a computer to modify the amplitude and intensity of said customized filter broadcast by said meridian linking device to vary the stress induced within said meridian network by said customized filter, said amplification function facilitating diagnosis of both manifested maladies needing corrective treatment and latent maladies eligible for preventative treatment.

19. The computer program product of claim 17, further comprising code that causes a computer to associate and assign one or more prescription constraints to said single, most effective and appropriate product/remedy.

20. The computer program product of claim 17, further comprising code that causes a computer to test the effectiveness and appropriateness of products/remedies that are not initially entered and stored within a product/remedy database against those said customized filters creating an imbalance within said meridian network.

21. The computer program product of claim 17, further comprising code that causes a computer to imprint a product/remedy and its associated electromagnetic signature onto or within an existing homeopathic remedy.

22. A computer readable medium comprising a data structure for storing patient remedies obtained by utilizing a computerized meridian linking diagnostic and treatment system that establishes and stabilizes one or more data access points as reference points and that links the meridians within a patient to form an interconnected meridian network that links all of the systems and organs within said patient to said one or more reference points, said remedies comprising one or more electromagnetic signatures corresponding to discovered treatments and treatment parameters.

23. A method in a computer system for identifying and treating both existing and latent maladies in a patient from a single reference data access point, said method comprising the steps of:
obtaining a computerized meridian linking diagnostic and treatment system;
activating a point stabilization function to broadcast a point stabilization filter that establishes one or more data access points as stable reference points having measured resistance values within a pre-determined stable range;
activating a meridian linking function to broadcast a meridian linking filter that forms a meridian network that links all of the organs and systems in a patient to a single stable reference point;
activating a filter testing function to broadcast a customized filter corresponding to a predetermined malady, said customized filter stresses said meridian network to diagnose any maladies in said patient;
amplifying said customized filter to vary its intensity, thus revealing latent maladies eligible for preventative treatment;
testing through one or more products/remedies automatically loaded by and stored within said system in response to any one of said customized filters that induce an imbalance in said meridian network to identify a single, most effective and appropriate remedy for treatment of said malady; and
administering said single, most effective and appropriate product/remedy to said patient.

24. The method of claim 23, further comprising assigning one or more prescription constraints to said single, most effective and appropriate remedy prior to said step of administering.

25. The method of claim 23, further comprising testing the effectiveness and appropriateness of products/remedies that are not initially entered and stored within a product/remedy database against those said customized filters creating an imbalance within said meridian network, and utilizing these as treatment remedies.

26. The method of claim 23, further comprising imprinting a product/remedy and its associated electromagnetic signature onto or within an existing homeopathic remedy to create a customized homeopathic remedy and administering said customized homeopathic remedy to said patient as treatment.
